(19) United States
(12) Patent Application Publication
(10) Pub. No.: US 2005/0147690 A1
(43) Pub. Date: Jul. 7, 2005

(54) Biocompatible protein particles, particle devices and methods thereof

(76) Inventors: David B. Masters, Minneapolis, MN (US); Eric P. Berg, Plymouth, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/962,984

(22) Filed: Oct. 12, 2004

Related U.S. Application Data
(63) Continuation-in-part of application No. 09/160,424, filed on Sep. 25, 1998.
(60) Provisional application No. 60/509,823, filed on Oct. 9, 2003.

(51) Int. Cl. A61K 9/127; A61K 9/50

(52) U.S. Cl. 424/499

(57) ABSTRACT

The present invention relates to biocompatible protein particles, particle devices and their methods of preparation and use. More specifically the present invention relates protein particles and devices derived from such particles comprising one or more biocompatible purified proteins combined with one or more biocompatible solvents. In various embodiments of the present invention the protein particles may also include one or more pharmacologically active agents and/or one or more additives.

### CROSS-REFERENCE TO RELATED APPLICATION

This application is a continuation in part of U.S. application Ser. No. 09/160,424 filed on Feb. 28, 2001. Furthermore, this patent claims priority to and incorporates by reference the entire contents of the previously mentioned application, U.S. application Ser. No. 09/922,418, filed on Aug. 3, 2001, and U.S. Provisional Application Ser. No. 60/509,823, filed on Oct. 9, 2003.

### FIELD OF THE INVENTION

The present invention relates to biocompatible protein particles, particle devices and their methods of preparation and use. More specifically the present invention relates protein particles and devices derived from such particles comprising one or more biocompatible purified proteins combined with one or more biocompatible solvents. In various embodiments of the present invention the protein particles may also include one or more pharmacologically active agents and/or one or more additives.

### BACKGROUND OF THE INVENTION

Protein materials are generally present in the tissues of many biological species. Therefore, the development of medical devices that utilize protein materials, which mimic and/or are biocompatible with the host tissue, have been pursued as desirable devices due to their acceptance and incorporation into such tissue. For example the utilization of protein materials to prepare drug delivery devices, tissue grafts, wound healing and other types of medical devices have been perceived as being valuable products due to their potential biocompatibility.

The use of dried protein, gelatins and/or hydrogels have previously been used as components for the preparation of devices for drug delivery, wound healing, tissue repair, medical device coating and the like. However, many of these previously developed devices do not offer sufficient strength, stability and support when administered to tissue environments that contain high solvent content, such as the tissue environment of the human body. Furthermore, the features of such medical devices that additionally incorporated pharmacologically active agents often provided an ineffective and uncontrollable release of such agents, thereby not providing an optimal device for controlled drug delivery.

A concern and disadvantage of such devices is the rapid dissolving or degradation of the device upon entry into an aqueous or high solvent environment. For example, gelatins and compressed dry proteins tend to rapidly disintegrate and/or lose their form when placed in an aqueous environment. Therefore, many dried or gelatin type devices do not provide optimal drug delivery and/or structural and durability characteristics. Also, gelatins often contain large amounts of water or other liquid that makes the structure fragile, non-rigid and unstable. Alternatively, dried protein devices are often very rigid, tend to be brittle and are extremely susceptible to disintegration upon contact with solvents. It is also noted that the proteins of gelatins usually denature during preparation caused by heating, the gelation process and/or crosslinking procedures, thereby reducing or eliminating the beneficial characteristics of the protein. The deficiencies gelatins and dried matrices have with regards to rapid degradation and structure make such devices less than optimal for the controlled release of pharmacologically active agents, or for operating as the structural scaffolding for devices such as vessels, stents or wound healing implants.

Hydrogel-forming polymeric materials, in particular, have been found to be useful in the formulation of medical devices, such as drug delivery devices. See, e.g., Lee, J Controlled Release, 2, 277 (1985). Hydrogel-forming polymers are polymers that are capable of absorbing a substantial amount of water to form elastic or inelastic gels. Many non-toxic hydrogel-forming polymers are known and are easy to formulate. Furthermore, medical devices incorporating hydrogel-forming polymers offer the flexibility of being capable of being implantable in liquid or gelled form. Once implanted, the hydrogel forming polymer absorbs water and swells. The release of a pharmacologically active agent incorporated into the device takes place through this gelled matrix via a diffusion mechanism.

However, many hydrogels, although biocompatible, are not biodegradable or are not capable of being remodeled and incorporated into the host tissue. Furthermore, most medical devices comprising of hydrogels require the use of undesirable organic solvents for their manufacture. Residual amounts of such solvents could potentially remain in the medical device, where they could cause solvent-induced toxicity in surrounding tissues or cause structural or pharmacological degradation to the pharmacologically active agents incorporated within the medical device. Finally, implanted medical devices that incorporate pharmacologically active agents in general, and such implanted medical devices comprising hydrogel-forming polymers in particular, oftentimes provide suboptimal release characteristics of the drug(s) incorporated therein. That is, typically, the release of pharmacologically active agents from an implanted medical device that includes pharmacologically active agent(s) is irregular, e.g., there is an initial burst period when the drug is released primarily from the surface of the device, followed by a second period during which little or no drug is released, and a third period during which most of the remainder of the drug is released or alternatively, the drug is released in one large burst.

Also, particles made from decellularized tissue, such as human, bovine or porcine tissue, have also been utilized in various medical applications. These decellularized tissue particles have been utilized in various applications as subcutaneous tissue fill materials. Futhermore, these substances have been shown to have some biocompatible properties, but generally are difficult to work with due to the already established matrix present in such materials. Furthermore, such tissue related materials are not conducive to the homogenous distribution of pharmacologically active agents within their matrix structure.

Additonally, other polymeric materials, such as polyvinyl pyrrolidone, polyvinyl alcohols, polyurethanes, polytetrafluoroethylene (PTFE), polypolyvinyl ethers, polyvinylidene halides, polyacrylonitrile, polyvinyl ketones; polyvinyl aromatics, ethylene-methyl methacrylate copolymers, polyamides, polycarbonates, polyoxymethylenes, polyimides, polyethers and other polymeric materials may be utilized as coatings for medical devices, drug delivery devices, tissue fillers or grafts, sutures and for other medical applications. These materials possess some biocompatible attributes, but are limited by there capacity to be non-thrombogenic, to be non-inflammatory, to allow direct cell integration, to deliver therapeutic agents, to allow regeneration of host tissue into the graft and/or to allow other graft materials to adhere to their surface.

### SUMMARY OF THE INVENTION

The protein particles of the present invention generally include one or more biocompatible proteins and one or more biocompatible solvents that are prepared at the proper composition to form a cohesive body. The cohesive body is next solidified into a compressed or spread matrix and processed into the particles of the present invention. Furthermore, embodiments of the protein particles of the present invention may also include one or more therapeutic pharmacologically active agents which are homogenously dispersed throughout each protein particle. Various embodiments of the protein particles of the present invention may also include a homogenous distribution of the protein, solvent and other additives, as well as the homogenous distribution of the pharmacologically active agents, to provide desired characteristics, such as drug elution control, durability, elasticity, strength and the like.

The biocompatible protein particles of the present invention are designed to retain the protein's natural activity combined with the ability to form it into various sized particles with structural integrity. The protein particles are further designed to compatibly mimic the host tissue composition and/or promote the remodeling of the particles into an architectural framework to support natural tissue growth. Generally, the protein particles of the present invention are biocompatible, biodegradable, and/or biointegratable thereby allowing the integration and remodeling of the particulate material by the host tissue. In addition to the ability to act as a structural scaffold, the ability to customize the material properties to the application, to mold the particles into any defined shape, and to incorporate other substances such as pharmacologically active agents (drugs), or other structural materials, into the protein particles also make the particles unique.

The foregoing and additional advantages and characterizing features of the present invention will become increasingly apparent to those of ordinary skill in the art by references to the following detailed description and to the drawings.

### BRIEF DESCRIPTION OF THE DRAWINGS

FIG. 1 depicts another embodiment of the particles of the present invention wherein the particles are porous;

FIG. 2 depicts one embodiment of the particles of the present invention sieved to between 75 and 125 microns;

FIG. 3 depicts one embodiment of a slurry of the present invention including particles in saline solution being passed through a syringe;

FIG. 4 depicts another embodiment of the present invention wherein the particles are compressed into a wafer form;

FIG. 5 depicts an embodiment of a biocompatible surface material comprising a polymeric base layer including a biocompatible surface of particles.

FIG. 6 depicts an embodiment of a protrusion device 34 that includes a port seal.

### DETAILED DESCRIPTION OF THE INVENTION

The biocompatible protein particles of the present invention are generally comprised of one or more biocompatible purified proteins and one or more biocompatible solvents. In various embodiments of the present invention, the protein particles may also include one or more pharmacologically active agents. It is noted that additional additive materials, such as biocompatible polymers like polyanhydride, polylactic acid, polyurethane and the like, and/or therapeutic entities may be included in the material to provide various beneficial features such as strength, elasticity, structure, enhanced biocompatibility and/or any other desirable characteristics. In various embodiments of the present invention, the particles possess a relatively homogeneous distribution of the components, including a homogenous distribution of any pharmacologically active agents and additive materials.

As previously mentioned, the biocompatible protein particles normally comprise one or more biocompatible purified synthetic proteins, genetically-engineered proteins, natural proteins or any combination thereof. In many embodiments of the present invention, the particles comprise a water-absorbing, biocompatible purified protein. The utilization of a water-absorbing biocompatible purified protein provides the advantage that, not only will the biocompatible protein particles be bioresorbable, but may remodel to mimic and support the tissue it contacts. That is, the metabolites of any degradation and/or resorption of the water-absorbing biocompatible purified protein may be reused by the patient's body rather than excreted.

Additionally, the proteins of the present invention are generally purified and in a free-form state. Normally, purified proteins are comprised of protein molecules that are not substantially crosslinked to other protein molecules, unlike tissues or gelatins. Normally, tissue or gelatin is already in a crosslinked matrix form and is thereby limited in forming new intermolecular or intramolecular bonds. Therefore, the purified protein molecules when added to solvent have the capacity to freely associate or intermingle with each other and other molecules or particles, such as solvents or pharmacologically active agents to form a homogeneous structure. Additionally, the binding sites of the purified free-form proteins for the attraction and retention of solvent, drug, protein or other molecules are generally available for binding whereas proteins derived from tissues and gelatins have generally lost some or most of its binding capability.

As previously suggested, the biocompatible purified protein utilized may either be naturally occurring, synthetic or genetically engineered. A preferred embodiment of the present invention includes insoluble naturally occurring purified protein. Naturally occurring purified protein that may be utilized in the protein particles of the present invention include, but are not limited to elastin, collagen, albumin, ovalbumin, keratin, fibronectin, vitronectin, laminin, thrombospondin, silk, silk fibroin, actin, myosin, fibrinogen, thrombin, aprotinin, antithrombin III, active proteins (e.g. interleukin, interferon, bone morphogenic protein (BMP) and the like), and any other biocompatible purified natural protein. Examples of purified proteins that are commercially available and may be utilized in some embodiments of the present invention include Type I insoluble collagen and insoluble elastin, manufactured by Kensey Nash Corporation, 55 East Uwchlan Avenue, Exton, Pa. 19341, Sigma-Aldrich Corporation, St. Louis, Mo., USA or Elastin Products Company, Inc., P.O. Box 568, Owensville, Mo., USA 65066. Other embodiments of the present invention may include soluble proteins. Examples of such soluble proteins include, but are not limited to Type I soluble collagen, soluble elastin, and soluble albumen manufactured by Kensey Nash Corporation, 55 East Uwchlan Avenue, Exton, Pa. 19341, Sigma-Aldrich Corporation, St. Louis, Mo., USA or Elastin Products Company, Inc., P.O. Box 568, Owensville, Mo., USA 65066. It is noted that combinations of purified natural proteins may be utilized to optimize desirable characteristics of the resulting biomatrix materials, such as strength, swelling, integration, cellular remodeling, degradability, resorption, drug absorption, etc. Inasmuch as heterogeneity in molecular weight, sequence and stereochemistry can influence the function of a protein in a biomatrix material, in some embodiments of the present invention synthetic or genetically engineered proteins are preferred in that a higher degree of control can be exercised over these parameters.

As previously suggested the proteins of the present invention are generally purified proteins. The purity of each natural protein component mixed in the coatable composition phase (the coatable composition will be described further below) during production of particles include 20% or less other proteins or impurities, preferably 10% or less other proteins or impurities, more preferably 3% or less other proteins or impurities and if available ideally 1% or less other proteins or impurities.

Synthetic proteins are generally prepared by chemical synthesis utilizing techniques known in the art. Also, individual proteins may be chemically combined with one or more other proteins of the same or different type to produce a dimer, trimer or other multimer. A simple advantage of having a larger protein molecule is that it will make interconnections with other protein molecules to create a stronger biomatrix material that is less susceptible to dissolving in aqueous solutions and provides additional protein structural and biochemical characteristics.

Additional, protein molecules can also be chemically combined to any other chemical so that the chemical does not release from the biocompatible protein particles. In this way, the chemical entity can provide surface modifications to particles or structural contributions to the particles to produce specific characteristics. The surface modifications can enhance and/or facilitate cell attachment depending on the chemical substance or the cell type. The structural modifications can be used to facilitate or impede dissolution, enzymatic degradation or dissolution of the particulate material.

Synthetic biocompatible purified proteins may be cross-linked, linked, bonded, chemically and/or physically linked to pharmacological active agents, enzymatically, chemically or thermally cleaved and utilized alone or in combination with other biocompatible proteins or partial proteins e.g. peptides, to form the biocompatible particles. Examples of such synthetic biocompatible proteins include, but are not limited to heparin-protein, heparin-protein-polymer, heparan sulfate-protein, heparan sulfate-polymer, heparan sulfate proteoglycans-protein, heparan sulfate proteoglycans-polymer, heparan sulfate-protein-polymer, chondroitin-protein, chondroitin-polymer, chondroitin-protein-polymer, chondroitin sulfate-protein, chondroitin sulfate-polymer, chondroitin sulfate-protein-polymer, heparan sulfate proteoglycans-cellulose, heparan sulfate proteoglycans-alginate, heparan sulfate proteoglycans-polylactide, GAGs-collagen, heparin-collagen, collagen-elastin-heparin, collagen-albumin, collagen-albumin-heparin, collagen-albumin-elastin-heparin, collagen-hyaluronic acid, collagen-chondroitin-heparin, collagen-chondroitin, derivatives thereof and the like.

A specific example of a particularly preferred genetically engineered protein for use in the biocompatible protein particles of the present invention is human collagen produced by FibroGen, Inc., 225 Gateway Blvd., South San Francisco, Calif. 94080. Other examples of particularly preferred genetically engineered proteins for use in the biocompatible protein particles of the present invention are commercially available under the nomenclature “ELP”, “SLP”, “CLP”, “SLPL”, “SLPF” and “SELP” from Protein Polymer Technologies, Inc. San Diego, Calif. ELP's, SLP's, CLP's, SLPL's, SLPF's and SELP's are families of genetically engineered protein polymers consisting of silklike blocks, elastinlike blocks, collagenlike blocks, lamininlike blocks, fibronectinlike blocks and the combination of silklike and elastinlike blocks, respectively. The ELP's, SLP's, CLP's, SLPL's, SLPF's and SELP's are produced in various block lengths and compositional ratios. Generally, blocks include groups of repeating amino acids making up a peptide sequence that occurs in a protein. Genetically engineered proteins are qualitatively distinguished from sequential polypeptides found in nature in that the length of their block repeats can be greater (up to several hundred amino acids versus less than ten for sequential polypeptides) and the sequence of their block repeats can be almost infinitely complex. Table A depicts examples of genetically engineered blocks. Table A and a further description of genetically engineered blocks may be found in Franco A. Ferrari and Joseph Cappello, Biosynthesis of Protein Polymers, in: Protein-Based Materials, (eds., Kevin McGrath and David Kaplan), Chapter 2, pp. 37-60, Birkhauser, Boston (1997).

TABLE A
Protein polymer sequences

| Polymer | |
|---|---|
| Name | Monomer Amino Acid Sequence |
| SLP 3 | [(GAGAGS)₉ GAAGY)] |
| SLP 4 | (GAGAGS)ₙ |
| SLP F | [(GAGAGS)₉ GAA VTGRGDSPAS AAGY]ₙ |
| SLP L3.0 | [(GAGAGS)₉ GAA PGASIKVAVSAGPS AGY]ₙ |
| SLP L3.1 | [(GAGAGS)₉ GAA PGASIKVAVSGPS AGY]ₙ |
| SLP F9 | [(GAGAGS)₉ RYVVLPRPVCFEK AAGY]ₙ |
| ELP I | [(VPGVG)₄]ₙ |
| SELP 0 | [(GVGVP)₈ (GAGAGS)₂]ₙ |
| SELP 1 | [GAA (VPGVG)₄ VAAGY (GAGAGS)₉]ₙ |
| SELP 2 | [(GAGAGS)₆ GAAGY (GAGAGS)₅ (GVGVP)₈]ₙ |
| SELP 3 | [(GVGVP)₈ (GAGAGS)₈]ₙ |
| SELP 4 | [(GVGVP)₁₂ (GAGAGS)₈]ₙ |
| SELP 5 | [(GVGVP)₁₆ (GAGAGS)₈]ₙ |
| SELP 6 | [(GVGVP)₃₂ (GAGAGS)₈]ₙ |
| SELP 7 | [(GVGVP)₈ (GAGAGS)₆]ₙ |
| SELP 8 | [(GVGVP)₈ (GAGAGS)₄]ₙ |
| KLP 1.2 | [(AKLKLAEAKLELAE)₄]ₙ |
| CLP 1 | [GAP(GPP)₄]ₙ |
| CLP 2 | {[GAP(GPP)₄]₂ GPAGPVGSP}ₙ |
| CLP-CB | {[GAP(GPP)₄]₂ (GLPGPKGDRGDAGPKGADGSPGPA) |
| | GPAGPVGSP}ₙ |
| CLP 3 | (GAPGAPGSQGAPGLQ)ₙ |

Repetitive amino acid sequences of selected protein polymers.
SLP = silk like protein;
SLPF = SLP containing the RGD sequence from fibronectin;
SLPL 3/0 and SLPL 3/1 = SLP containing two difference sequences from laminin protein;
ELP = elastin like protein;
SELP = silk elastin like protein;
CLP = collagen like protein;
CLP-CB = CLP containing a cell binding domain from human collagen;
KLP = keratin like protein

The nature of the elastinlike blocks, and their length and position within the monomers influences the water solubility of the SELP polymers. For example, decreasing the length and/or content of the silklike block domains, while maintaining the length of the elastinlike block domains, increases the water solubility of the polymers. For a more detailed discussion of the production of SLP's, ELP's, CLP's, SLPF's and SELP's as well as their properties and characteristics see, for example, in J. Cappello et al., Biotechnol. Prog., 6, 198 (1990), the full disclosure of which is incorporated by reference herein. One preferred SELP, SELP7, has an elastin:silk ratio of 1.33, and has 45% silklike protein material and is believed to have weight average molecular weight of 80,338.

Generally, the amount of purified protein found in embodiments of the particles of the present invention may vary between from about 15% to about 85%, preferably from about 20% to 80% by weight, and most preferably from about 50% to 70% by weight based upon the weight of the final particles. As used herein, unless stated otherwise, all percentages are percentages based upon the total mass of the composition or particles being described, e.g., 100% is total.

The biocompatible protein particles utilized in various embodiments of the present invention also include one or more biocompatible solvents. Any biocompatible solvent may be utilized in the method and corresponding biomatrix material of the present invention. By using a biocompatible solvent, the risk of adverse tissue reactions to residual solvent remaining in the device after manufacture is minimized. Additionally, the use of a biocompatible solvent reduces the potential structural and/or pharmacological degradation of the pharmacologically active agent that some such pharmacologically active agents undergo when exposed to organic solvents. Suitable biocompatible solvents for use in the method of the present invention include, but are not limited to, water; dimethyl sulfoxide (DMSO); simple biocompatible alcohols, such as methanol and ethanol; various acids, such as formic acid; oils, such as olive oil, peanut oil and the like; ethylene glycol, glycols; and combinations of these and the like. Preferably, the biocompatible solvent comprises water. The amount of biocompatible solvent utilized in the coatable composition will preferably be that amount sufficient to result in the composition being fluid and flowable enough to be coatable. Generally, the amount of biocompatible solvent suitable for use in the method of the present invention will range from about 50% to about 1000%, preferably from about 100% to about 300% by weight, based upon the weight and/or amount of the protein utilized.

In addition to the biocompatible protein(s) and the biocompatible solvent(s), the biocompatible protein particles that may be utilized in various embodiments of the present invention may include one or more pharmacologically active agents. As used herein, “pharmacologically active agent” generally refers to a pharmacologically active agent having a direct or indirect beneficial therapeutic effect upon introduction into a host. Pharmacologically active agents further include neutraceuticals. The phrase “pharmacologically active agent” is also meant to indicate prodrug forms thereof. A “prodrug form” of a pharmacologically active agent means a structurally related compound or derivative of the pharmacologically active agent which, when administered to a host is converted into the desired pharmacologically active agent. A prodrug form may have little or none of the desired pharmacological activity exhibited by the pharmacologically active agent to which it is converted. Representative examples of pharmacologically active agents that may be suitable for use in the particles and particle devices of the present invention include, but are not limited to, (grouped by therapeutic class):

Antidiarrheals such as diphenoxylate, loperamide and hyoscyamine;

Antihypertensives such as hydralazine, minoxidil, captopril, enalapril, clonidine, prazosin, debrisoquine, diazoxide, guanethidine, methyldopa, reserpine, trimethaphan;

Calcium channel blockers such as diltiazem, felodipine, amlodipine, nitrendipine, nifedipine and verapamil;

Antiarrhyrthmics such as amiodarone, flecainide, disopyramide, procainamide, mexiletene and quinidine,

Antiangina agents such as glyceryl trinitrate, erythrityl tetranitrate, pentaerythritol tetranitrate, mannitol hexanitrate, perhexilene, isosorbide dinitrate and nicorandil;

Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate;

Cardiotonic glycosides such as digoxin and other cardiac glycosides and theophylline derivatives;

Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine;

Vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol;

Antiproliferative agents such as paclitaxel, estradiol, actinomycin D, sirolimus, tacrolimus, everolimus and dexamethasone;

Antimigraine preparations such as ergotanmine, dihydroergotamine, methysergide, pizotifen and sumatriptan;

Anticoagulants and thrombolytic agents such as warfarin, dicoumarol, low molecular weight heparins such as enoxaparin, streptokinase and its active derivatives;

Hemostatic agents such as aprotinin, tranexamic acid and protamine;

Analgesics and antipyretics including the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine dihydrocodeine; acetylsalicylic acid (aspirin), paracetamol, and phenazone;

Immunosuppressants, antiproliferatives and cytostatic agents such as rapomycin (sirolimus) and its analogs (everolimus and tacrolimus);

Neurotoxins such as capsaicin, botulinum toxin (botox);

Hypnotics and sedatives such as the barbiturates amylobarbitone, butobarbitone and pentobarbitone and other hypnotics and sedatives such as chloral hydrate, chlormethiazole, hydroxyzine and meprobamate;

Antianxiety agents such as the benzodiazepines alprazolam, bromazepam, chlordiazepoxide, clobazam, chlorazepate, diazepam, flunitrazepam, flurazepam, lorazepam, nitrazepam, oxazepam, temazepam and triazolam;

Neuroleptic and antipsychotic drugs such as the phenothiazines, chlorpromazine, fluphenazine, pericyazine, perphenazine, promazine, thiopropazate, thioridazine, trifluoperazine; and butyrophenone, droperidol and haloperidol; and other antipsychotic drugs such as pimozide, thiothixene and lithium;

Antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline;

CNS stimulants such as caffeine and 3-(2-aminobutyl)indole;

Anti-alzheimer's agents such as tacrine;

Anti-Parkinson's agents such as amantadine, benserazide, carbidopa, levodopa, benztropine, biperiden, benzhexol, procyclidine and dopamine-2 agonists such as S (−)-2-(N-propyl-N-2-thienylethylamino)-5-hydroxytetralin (N-0923),

Anticonvulsants such as phenytoin, valproic acid, primidone, phenobarbitone, methylphenobarbitone and carbamazepine, ethosuximide, methsuximide, phensuximide, sulthiame and clonazepam,

Antiemetics and antinauseants such as the phenothiazines prochloperazine, thiethylperazine and 5HT-3 receptor antagonists such as ondansetron and granisetron, as well as dimenhydrinate, diphenhydramine, metoclopramide, domperidone, hyoscine, hyoscine hydrobromide, hyoscine hydrochloride, clebopride and brompride;

Non-steroidal anti-inflammatory agents including their racemic mixtures or individual enantiomers where applicable, preferably which can be formulated in combination with dermal and/or mucosal penetration enhancers, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate;

Antirheumatoid agents such as penicillamine, aurothioglucose, sodium aurothiomalate, methotrexate and auranofin;

Muscle relaxants such as baclofen, diazepam, cyclobenzaprine hydrochloride, dantrolene, methocarbamol, orphenadrine and quinine;

Agents used in gout and hyperuricaemia such as allopurinol, colchicine, probenecid and sulphinpyrazone;

Oestrogens such as oestradiol, oestriol, oestrone, ethinyloestradiol, mestranol, stilboestrol, dienoestrol, epioestriol, estropipate and zeranol;

Progesterone and other progestagens such as allyloestrenol, dydrgesterone, lynoestrenol, norgestrel, norethyndrel, norethisterone, norethisterone acetate, gestodene, levonorgestrel, medroxyprogesterone and megestrol;

Antiandrogens such as cyproterone acetate and danazol;

Antioestrogens such as tamoxifen and epitiostanol and the aromatase inhibitors, exemestane and 4-hydroxy-androstenedione and its derivatives;

Androgens and anabolic agents such as testosterone, methyltestosterone, clostebol acetate, drostanolone, furazabol, nandrolone oxandrolone, stanozolol, trenbolone acetate, dihydro-testosterone, 17-(α-methyl-19-noriestosterone and fluoxymesterone;

5-alpha reductase inhibitors such as finasteride, turosteride, LY-191704 and MK-306;

Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate, methylprednisolone, prednisolone, prednisolone 21-phosphate, prednisone, triamcinolone, triamcinolone acetonide;

Glycosylated proteins, proteoglycans, glycosaminoglycans and bio-mimic agents such as heparin, heparan-sulfate, chondroitin sulfate; chitin, acetyl-glucosamine, hyaluronic acid keratin sulfate and dermatin sulfate;

Complex carbohydrates such as glucans;

Further examples of steroidal anti-inflammatory agents such as cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, cortodoxone, flucetonide, fludrocortisone acetate, flurandrenolone, aincinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol;

Pituitary hormones and their active derivatives or analogs such as corticotrophin, thyrotropin, follicle stimulating hormone (FSH), luteinising hormone (LH) and gonadotrophin releasing hormone (GnRH), growth hormone;

Hypoglycemic agents such as insulin, chlorpropamide, glibenclamide, gliclazide, glipizide, tolazamide, tolbutamide and metformin;

Thyroid hormones such as calcitonin, thyroxine and liothyronine and antithyroid agents such as carbimazole and propylthiouracil;

Other miscellaneous hormone agents such as octreotide;

Pituitary inhibitors such as bromocriptine;

Ovulation inducers such as clomiphene;

Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potasium sparing diuretics, spironolactone, amiloride and triamterene;

Antidiuretics such as desmopressin, lypressin and vasopressin including their active derivatives or analogs;

Obstetric drugs including agents acting on the uterus such as ergometrine, oxytocin and gemeprost;

Prostaglandins such as alprostadil (PGE1), prostacyclin (PGI2), dinoprost (prostaglandin F2-alpha) and misoprostol;

Antimicrobials including the cephalosporins such as cephalexin, cefoxytin and cephalothin;

Penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, meziocillin, piperacillin, ticarcillin and azlocillin;

Tetracyclines such as minocycline, chlortetracycline, tetracycline, demeclocycline, doxycycline, methacycline and oxytetracycline and other tetracycline-type antibiotics;

Aminoglycoides such as amikacin, gentamicin, kanamycin, neomycin, netilmicin and tobramycin;

Antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione;

Quinolones such as nalidixic acid, cinoxacin, ciprofloxacin, enoxacin and norfloxacin;

Sulphonamides such as phthalysulphthiazole, sulfadoxine, sulphadiazine, sulphamethizole and sulphamethoxazole;

Sulphones such as dapsone;

Other miscellaneous antibiotics such as cyclosporin, chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonam, colistin IV, metronidazole, tinidazole, fusidic acid, trimethoprim, and 2-thiopyridine N-oxide; halogen compounds, particularly iodine and iodine compounds such as iodine-PVP complex and diiodohydroxyquin, hexachlorophene; chlorhexidine; chloroamine compounds; and benzoylperoxide;

Antituberculosis drugs such as ethambutol, isoniazid, pyrazinamide, rifampicin and clofazimine;

Antimalarials such as primaquine, pyrimethamine, chloroquine, hydroxychloroquine, quinine, mefloquine and halofantrine;

Antiviral agents such as acyclovir and acyclovir prodrugs, famcyclovir, zidovudine, didanosine, stavudine, lamivudine, zalcitabine, saquinavir, indinavir, ritonavir, n-docosanol, tromantadine and idoxuridine;

Anthelmintics such as mebendazole, thiabendazole, niclosamide, praziquantel, pyrantel embonate and diethylcarbamazine;

Cytotoxic agents such as plicamycin, cyclophosphamide, dacarbazine, fluorouracil and its prodrugs (described, for example, in International Journal of Pharmaceutics, 111, 223-233 (1994)), methotrexate, procarbazine, 6-mercaptopurine and mucophenolic acid;

Anorectic and weight reducing agents including dexfenfluramine, fenfluramine, diethylpropion, mazindol and phentermine;

Agents used in hypercalcaemia such as calcitriol, dihydrotachysterol and their active derivatives or analogs;

Antitussives such as ethylmorphine, dextromethorphan and pholcodine;

Expectorants such as carbolcysteine, bromhexine, emetine, quanifesin, ipecacuanha and saponins;

Decongestants such as phenylephrine, phenylpropanolamine and pseudo ephedrine;

Bronchospasm relaxants such as ephedrine, fenoterol, orciprenaline, rimiterol, salbutamol, sodium cromoglycate, cromoglycic acid and its prodrugs (described, for example, in International Journal of Pharmaceutics 7, 63-75 (1980)), terbutaline, ipratropium bromide, salmeterol and theophylline and theophylline derivatives;

Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine;

Local anaesthetics such as benzocaine, bupivacaine, amethocaine, lignocaine, lidocaine, cocaine, cinchocaine, dibucaine, mepivacaine, prilocalne, etidocaine, veratridine (specific c-fiber blocker) and procaine;

Stratum corneum lipids, such as ceramides, cholesterol and free fatty acids, for improved skin barrier repair [Man, et al. J. Invest. Dermatol, 106(5), 1096, (1996)];

Neuromuscular blocking agents such as suxamethonium, alcuronium, pancuronium, atracurium, gallamine, tubocurarine and vecuronium;

Smoking cessation agents such as nicotine, bupropion and ibogaine;

Insecticides and other pesticides which are suitable for local application;

Dermatological agents, such as vitamins A, C, B1, B2, B6, B12, B12a., and E, vitamin E acetate and vitamin E sorbate;

Allergens for desensitisation such as house, dust or mite allergens;

Nutritional agents and neutraceuticals, such as vitamins, essential amino acids and fats;

Macromolecular pharmacologically active agents such as proteins, enzymes, peptides, polysaccharides (such as cellulose, amylose, dextran, chitin), nucleic acids, cells, tissues, and the like;

Bone mending biochemicals such as calcium carbonate, calcium phosphate, hydroxyapetite or bone morphogenic protein (BMP);

Angiogenic growth factors such as Vascular Endothelial Growth Factor (VEGF) and epidermal growth factor (EGF), cytokines, interleukins, fibroblasts and cytotaxic chemicals, platelet derived growth factor (PDGF), fibroblast growth factor (FGF), tissue/wound healing growth factors; and

Keratolytics such as the alpha-hydroxy acids, glycolic acid and salicylic acid; and DNA, RNA or other oligonucleotides.

Permeation enhancers (e.g. membrane permeation enhancers) such as ascorbic acid, citric acid, glutamine and Lauroylcarnitine

Additionally, the biocompatible protein particles of the present invention are particularly advantageous for the encapsulation, incorporation and/or scaffolding of macromolecular pharmacologically active agents such as proteins, enzymes, peptides, polysaccharides, nucleic acids, cells, tissues, and the like. Immobilization of macromolecular pharmacologically active agents into or onto a particle can be difficult due to the ease with which some of these macromolecular agents denature when exposed to organic solvents, some constituents present in bodily fluids or to temperatures appreciably higher than room temperature. However, since the method of the present invention utilizes biocompatible solvents such as water, DMSO or ethanol the risk of the denaturation of these types of materials is reduced. Furthermore, due to the size of these macromolecular pharmacologically active agents, these agents may be encapsulated within the particles of the present invention and thereby are protected from constituents of bodily fluids that would otherwise denature them. Thus, the particles of the present invention allow these macromolecular agents to exert their therapeutic effects, while yet protecting them from denaturation or other structural degradation.

Examples of cells which can be utilized as the pharmacologically active agent in the biocompatible protein particles of the present invention include primary cultures as well as established cell lines, including transformed cells. Examples of these include, but are not limited to pancreatic islet cells, human foreskin fibroblasts, Chinese hamster ovary cells, beta cell insulomas, lymphoblastic leukemia cells, mouse 3T3 fibroblasts, dopamine secreting ventral mesencephalon cells, neuroblastold cells, adrenal medulla cells, endothelial cells, T-cells combinations of these, and the like. As can be seen from this partial list, cells of all types, including dermal, neural, blood, organ, stem, muscle, glandular, reproductive and immune system cells, as well as cells of all species of origin, can be encapsulated successfully by this method.

Examples of proteins which can be incorporated into the biocompatible protein particles of the present invention include, but are not limited to, hemoglobin, glutamic acid decarboxylase, vasporessin, oxytocin, adrenocorticocotrophic hormone, epidermal growth factor, prolactin, luliberin or luteinising hormone releasing factor, human growth hormone, and the like; enzymes such as adenosine deaminase, tyrosine hydroxylase, alcohol dehydrogenase, superoxide dismutase, xanthine oxidase, and the like; enzyme systems; blood clotting factors; clot inhibitors or clot dissolving agents such as streptokinase and tissue plasminogen activator; antigens for immunization; hormones; polysaccharides such as heparin, chondroitin sulfate and hyaluronic acid; oligonucleotides; bacteria and other microbial microorganisms including viruses; monoclonal antibodies, such as herceptin and rituximab; vitamins; cofactors; growth factors; retroviruses for gene therapy, combinations of these and the like.

An efficacious amount of the aforementioned pharmacologically active agent(s) can easily be determined by those of ordinary skill in the art taking into consideration such parameters as the particular pharmacologically active agent chosen, the size and weight of the patient, the desired therapeutic effect, the pharmacokinetics of the chosen pharmacologically active agent, and the like, as well as by reference to well known resources such as Physicians' Desk Reference®: PDR-52 ed (1998)—Medical Economics 1974. In consideration of these parameters, it has been found that a wide range exists in the amount of the pharmacologically active agent(s) capable of being incorporated into and subsequently released from or alternatively allowed to exert the agent's therapeutic effects from within the protein particles. More specifically, the amount of pharmacologically active agent that may be incorporated into and then either released from or active from within the biocompatible protein particles may range from about 0.001% to about 200%, more preferably, from about 0.05% to about 100%, most preferably from about 0.1% to 70%, based on the weight of the particulate material. It is important to note that the pharmacologically active agents are generally homogenously distributed throughout the particulate material thereby allowing for a controlled release of these agents.

Finally, one or more additive materials may be added to the coatable composition to manipulate the material properties and thereby add additional structure or modify the release of pharmacologically active agents. That is, while a particulate material that includes a relatively fast-degrading protein material without a particular additive material will readily degrade thereby releasing drug relatively quickly upon insertion or implantation, a particulate material that includes a particular polymeric material, such as polyanhydride, will degrade slowly, as well as release the pharmacologically active agent(s) over a longer period of time. Additionally, the addition of other additive materials, such as humectants like glycerin, pectin, polyethylene glycol, sorbitol, maltitol, mannitol, hydrogenated glucose syrups, xylitol, polydextrose, glyceryl triacetate and propylene glycol, may provide enhanced adhesion properties to parts of the body, such as mucosal tissue. Examples of biodegradable and/or biocompatible additive materials suitable for use in the biocompatible protein particles of the present invention include, but are not limited to polyurethanes, vinyl homopolymers and copolymers, acrylate homopolymers and copolymers, polyethers, cellulosics, epoxies, polyesters, acrylics, nylons, silicones, polyanhydride, poly(ethylene terephthalate), polyacetal, poly(lactic acid), poly(ethylene oxide)/poly(butylene terephthalate) copolymer, polycarbonate, poly(tetrafluoroethylene) (PTFE), polycaprolactone, polyethylene oxide, polyethylene glycol, poly(vinyl chloride), polylactic acid, polyglycolic acid, polypropylene oxide, poly(akylene)glycol, polyoxyethylene, sebacic acid, polyvinyl alcohol (PVA), 2-hydroxyethyl methacrylate (HEMA), polymethyl methacrylate, 1,3-bis(carboxyphenoxy)propane, lipids, phosphatidylcholine, triglycerides, polyhydroxybutyrate (PHB), polyhydroxyvalerate (PHV), poly(ethylene oxide) (PEO), poly ortho esters, poly(amino acids), polycynoacrylates, polyphophazenes, polysulfone, polyamine, poly(amido amines), fibrin, glycerin, pectin, sorbitol, maltitol, mannitol, hydrogenated glucose syrups, xylitol, polydextrose, glyceryl triacetate, propylene glycol, graphite, flexible fluoropolymer, isobutyl-based, isopropyl styrene, vinyl pyrrolidone, cellulose acetate dibutyrate, silicone rubber, copolymers of these, and the like. Other materials that may be incorporated into the coatable composition to provide enhanced features include, but are not limited to, ceramics, bioceramics, glasses bioglasses, glass-ceramics, resin cement, resin fill; more specifically, glass ionomer, hydroxyapatite, calcium sulfate, Al₂O₃, tricalcium phosphate, calcium phosphate salts, sugars, starches, carbohydrates, salts, polysaccharides, alginate and carbon. Additional other materials that may be incorporated into the coatable composition include alloys such as, cobalt-based, galvanic-based, stainless steel-based, titanium-based, zirconium oxide, zirconia, aluminum-based, vanadium-based, molybdenum-based, nickel-based, iron-based, or zinc-based (zinc phosphate, zinc polycarboxylate).

Other additives may be utilized, for example, to facilitate the processing of the biocompatible protein particles, to stabilize the pharmacologically active agents, to facilitate the activity of the pharmacologically active agents, or to alter the release characteristics of the biocompatible protein particles. For example, when the pharmacologically active agent is to be an enzyme, such as xanthine oxidase or superoxide dismutase, the protein matrix device may further comprise an amount of an enzyme substrate, such as xanthine, to facilitate the action of the enzyme.

Additionally, hydrophobic substances such as lipids can be incorporated into the biocompatible protein particles to extend the duration of drug release, while hydrophilic, polar additives, such as salts and amino acids, can be added to facilitate, i.e., shorten the duration of, drug release. Exemplary hydrophobic substances include lipids, e.g., tristeafin, ethyl stearate, phosphotidycholine, polyethylene glycol (PEG); fatty acids, e.g., sebacic acid erucic acid; combinations of these and the like. A particularly preferred hydrophobic additive useful to extend the release of the pharmacologically active agents comprises a combination of a dimer of erucic acid and sebacic acid, wherein the ratio of the dimer of erucic acid to sebacic acid is 1:4. Exemplary hydrophilic additives useful to shorten the release duration of the pharmacologically active agent include but are not limited to, salts, such as sodium chloride; and amino acids, such as glutamine and glycine. If additives are to be incorporated into the coatable composition, they will preferably be included in an amount so that the desired result of the additive is exhibited.

One method of producing the biocompatible protein particles of the present invention is by providing one or more selected biocompatible purified proteins, adding other materials (pharmacologically active agents, additives, etc.) and solvents (water) to form a coatable composition. Once prepared, the coatable composition may be coated onto any suitable surface from which it may be released after drying by any suitable method. Examples of suitable coating techniques include spin coating, gravure coating, flow coating, spray coating, coating with a brush or roller, screen printing, knife coating, curtain coating, slide curtain coating, extrusion, squeegee coating, and the like. The coated film (preferably having a substantially planar body having opposed major surfaces) is desirably thin enough so as to be capable of drying within a reasonable amount of time and also thin enough so that the film can be formed into a cohesive body comprising a substantially homogeneous dispersion of the components of the coatable composition. For example, a thinner film will tend to form a more homogeneous cohesive body when the film is formed into the shape of a cylinder. A typical coated film of the coatable composition have a thickness in the range of from about 0.01 millimeters to about 5 millimeters, more preferably from about 0.05 millimeters to about 2 millimeters.

Initially, when the film is first coated, it is likely to be non-cohesive, fluidly-flowable, and/or non self-supporting. Thus, the coated film is preferably dried sufficiently so that it becomes cohesive, i.e., the film preferably sticks to itself rather than other materials. The film may simply be allowed to dry at room temperature, or alternatively, may be dried under vacuum, conditions of mild heating, i.e., heating to a temperature of from about 25° C. to about 150° C., or conditions of mild cooling, i.e. cooling to a temperature of from about 0° C. to about 20° C. When utilizing heat to dry the film, care should be taken to avoid denaturation or structural degradation of the pharmacologically active agent incorporated therein. Also, care should be taken to not irreversibly denature the proteins of the cohesive body during preparation through various actions on the composition that will disrupt the secondary and/or tertiary structure of the protein(s) such as application of excessive heat or strong alkaline solution, which may cause coagulation/gelation. It is noted that the cohesive body may be prepared without the film step if the proper amounts of protein, solvent and other components are known to achieve the necessary characteristics of the cohesive body.

The specific solvent content at which the film and/or the composition becomes cohesive unto itself will depend on the individual components incorporated into the coatable composition. A cohesive body is achieved when the components of the composition are in the proper amounts so that the resulting composition is tacky or cohesive to itself more than to other materials or surface that it contacts. Generally, films that have too high of a solvent content will not be cohesive. Films that have too low of a solvent content will tend to crack, shatter, or otherwise break apart upon efforts to form them into a cohesive body. With these considerations in mind, the solvent content of a partially dried film will preferably be from about 10% to about 80%, more preferably from about 15% to about 65% and most preferably from about 20% to about 50%.

Once the film is capable of forming a cohesive body, such a cohesive body may be formed by any of a number of methods. For example, the film may be rolled, folded, accordion-pleated, crumpled, or otherwise shaped such that the resulting cohesive body has a surface area that is less than that of the coated film. For example the film can be shaped into a cylinder, a cube, a sphere or the like. Preferably, the cohesive body is formed by rolling the coated film to form a cylinder.

Additionally, embodiments of the present invention may include the addition of reagents to properly pH the resulting biocompatible protein particles and thereby enhance the biocompatible characteristics of the device with the host tissue of which it is to be administered. When preparing the biocompatible protein materials, the pH steps of the mixture of biocompatable materials, such as purified proteins, pharmacologically active agents and other additives, and the biocompatable solvent(s) occur prior to the partial drying preparation of the cohesive body. The pH steps can be started with the addition of biocompatable solvent to the protein or to the mixture of protein material and optional biocompatible materials, or the pH steps can be started after mixing the material(s) and solvent(s) together before the cohesive body is formed. For example, the pH steps can include the addition of drops of 0.05N to 4.0N acid or base to the solvent wetted material until the desired pH is reached as indicated by a pH meter, pH paper or any pH indicator. More preferably, the addition of drops of 0.1N-0.5 N acid or base are used. Although any acid or base may be used, the preferable acids and bases are HCl and NaOH, respectively. If known amounts of biocompatable material are used it may be possible to add acid or base to adjust the pH when the biocompatable material is first wetted, thereby allowing wetting and pH adjustments to occur in one step.

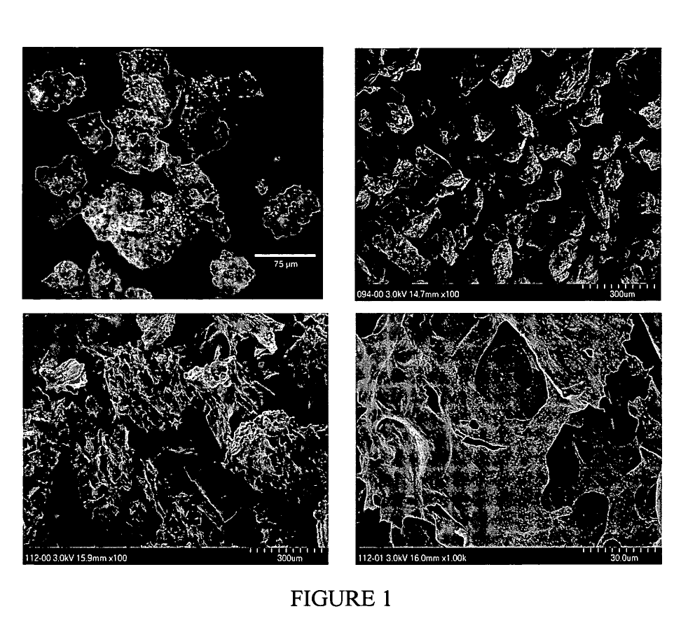

Furthermore, the cohesive body and/or particles may be set up with pores that allow fluid flow through that particles and also enhances movement of the pharmacologically active agents through the particles. Pores may be created in the cohesive body or particles by incorporating a substance in the cohesive body during its preparation that may be removed or dissolved out of the matrix before administration of the device or shortly after administration. Porosity may be produced in particles by the utilization of materials such as, but not limited to, salts such as NaCl, amino acids such as glutamine, microorganisms, enzymes, copolymers or other materials, which will be leeched out of the protein matrix to create pores. FIG. 1 depicts one embodiment of the present invention, wherein glutamine was included in the cohesive body and then dissolved out during crosslinking to form pores in the particles. Other functions of porosity are that the pores create leakage so that cells on outside can receive fluids that include the contents of the particles and also that cells may enter the particles to interact and remodel the matrix material to better incorporate and function within the host tissue.

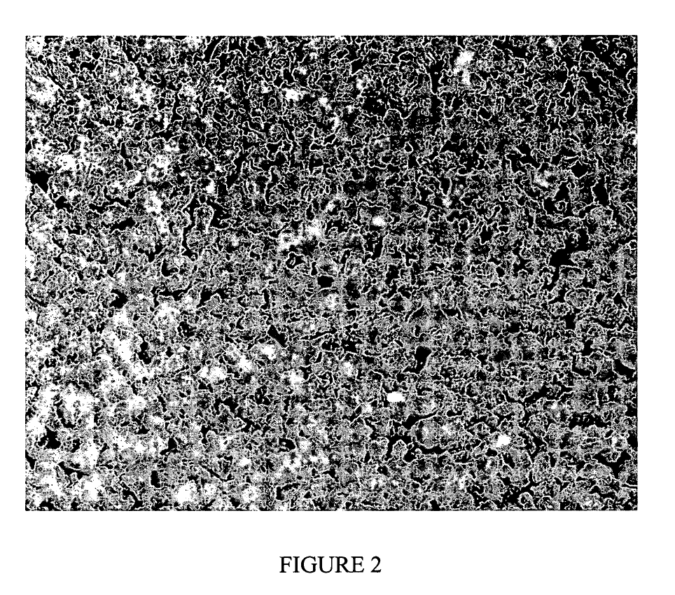

Once so formed, the cohesive body may be solidified prior to particle processing. The cohesive may be solidified into a compressed matrix or spread matrix form. A spread matrix form is generally solidifying the cohesive body utilizing one or more of solidifying techniques without applying compression to the cohesive body. It is noted that a combination of these techniques may also be utilized. Alternatives to solidify the cohesive body other than compression may be to apply heat, freeze drying, freezing to freeze fracture (e.g. liquid nitrogen, dry ice or conventional freezing) or other drying techniques to solidify the cohesive body before processing the cohesive body into particles. An illustration of one embodiment of particles of the present invention comprising collagen, elastin and heparin at a ratio of 7/2/1 is depicted in FIG. 2.

As previously suggested, particles may be derived from a biocompatible protein material produced by solidifying the cohesive body by applying heat, crosslinking, freeze fracturing techniques such as liquid nitrogen freeze fracturing or dry ice freeze drying, vacuum or other similar drying techniques to eliminate excess solvent from the cohesive body rather than compressing it. These alternative techniques remove enough solvent from the cohesive body to provide for the production of distinct particles, but do not eliminate too much solvent wherein the interaction of solvent and protein is lost. Generally, the proteins, solvent and optionally the pharmacologically active agents will interact by binding through intermolecular and intramolecular forces (i.e., ionic, dipole-dipole such as hydrogen bonding, London dispersion, hydrophobic, etc.) that are created during the steps of forming a cohesive body and then also when further solidifying the cohesive body.

One example of an alternative method to solidify the cohesive body to make particles is by heating the cohesive body and then processing the resulting solidified cohesive body into particles. In such a method the cohesive body may be heated at temperatures ranging from 0°-150° C., preferably 20°-120° C. and most preferably 40°-10⁰° C. Generally, the heating process may be conducted for approximately 15 seconds to 48 hours, preferably 20 seconds to 10 and most preferably 30 seconds to 1 hour. Embodiments of the resulting cohesive body following heating, or any of the alternative techniques identified above, usually have as little solvent as possible while still being cohesive and possessing the desired features relevant to the device's function, e.g., preferably a solvent content of from about 5% to about 60%, more preferably a solvent content of from about 10% to about 50% and most preferably 20% to 40%.

It is found that when a solidified cohesive body utilized in the production of the particles of the present invention includes one or more pharmacologically active agent, the partial drying of the film to form a cohesive body and subsequent solidification of the cohesive body, forces more solvent out of the body, thereby producing a resulting material that has a significantly higher concentration of pharmacologically active agents. As a result of the substantially uniform dispersion of a greater concentration of pharmacologically active agents, a sustained, controlled release of the pharmacologically active agent is achieved, while reducing the initial high concentration effects that can be associated with other devices that include pharmacologically active agents.

The cohesive body may also be solidified by compressing the cohesive body. For example, the cohesive body may be formed into a cylinder by compression that may be subsequently pulverized into particles (an explanation of methods to make particles is described below).

Any manually or automatically operable mechanical, pneumatic, hydraulic, or electrical molding device capable of subjecting the cohesive body to pressure is suitable for use in the method of the present invention. In the production of various embodiments of the present invention, a molding device may be utilized that is capable of applying a pressure of from about 100 pounds per square inch (psi) to about 100,000 psi for a time period of from about 0.2 seconds to about 48 hours. Preferably, the molding device used in the method of the present invention will be capable of applying a pressure of from about 1000 psi to about 30,000 psi for a time period of from about 0.5 second to about 60 minutes. More preferably, the molding device used in the method of the present invention will be capable of applying a pressure of from about 3,000 psi to about 25,000 psi for a time period of from about 1 second to about ten minutes.

Compression molding devices suitable for use in the practice of the method of the present invention are generally known. Suitable devices may be manufactured by a number of vendors according to provided specifications, such as desirable pressure, desired materials for formulation, desired pressure source, desired size of the moldable and resulting molded device, and the like. For example, Gami Engineering, located in Mississauga, Ontario manufactures compression molding devices to specifications provided by the customer. Additionally, many compression molding devices are commercially available. See U.S. Pat. No. 6,342,250 and U.S. application Ser. No. 09/796,170, which are incorporated by reference herein, for a description of compression molding devices that may be utilized in the process of the present invention and methods utilized to produce a compressed protein matrix.

Before the cohesive body is processed into particles or after particles are produced, the materials may also be crosslinked to provide additional beneficial characteristics. The optional step of crosslinking the cohesive body or particles may be performed by any means known in the art such as exposure to chemical crosslinking agents like glutaraldehyde, formaldehyde, p-Azidobenzolyl Hydazide, N-5-Azido 2-nitrobenzoyloxysuccinimide, glycidyl ethers such as 1,4-butandiol diglycidylether, N-Succinimidyl 6-[4′azido-2′nitro-phenylamino]hexanoate and 4-[p-Azidosalicylamido]butylamine, ultraviolet light or other radiation sources like ultrasound or gamma ray. Furthermore, it is also noted that multiple applications of one or more crosslinking agents at different stages may produce desired products. For example, crosslinking the cohesive body after initial formation and then again following homogenization or grinding of the cohesive body into particles has proven effective.

The particles of the present invention are generally prepared by further processing the solidified cohesive body. In various embodiments of the present invention, the particles are produced by further processing the cohesive body that has been solidified by the alternative methods described above. Various methods may be utilized to produce the particles of the present invention. Examples of methods of producing the particles of the present invention includes crushing, cutting, pulverizing, homogenizing or grinding of the solidified cohesive body in either wet or dry conditions until the particles are formed. These methods of producing the particles utilized in products of the present invention may be performed following the freezing of the cohesive body in liquid nitrogen, by utilizing other freeze/solid fracture or particle forming techniques or by partially heating the cohesive body until substantially rigid, but still retaining some solvent content.

In two embodiments of the present invention the particles are prepared utilizing a mill grinder or a homogenizer. Types of mill grinders and homogenizers that may be utilized include, but are not limited to ball mills, grinder stations, polytron homogeneizers and the like. One example of a polytron homogenizer that may be utilized in processing particles of the present invention may be a Polytron PT1200E purchased from the Kinematica corporation of Switzerland. An example of a ball mill that may be utilized in processing particles of the present invention may be a ballmill/rollermill purchased from U.S. Stoneware, Inc. and distributed by ER Advanced Ceramincs of Palestine, Ohio.

Generally, the particles may vary in size but are normally equal to or less than 2 mm. In many embodiments of the present invention the particles are approximately 10 nm-1.75 mm, preferably 500 nm-1.5 mm and more preferably 1-1000 μm. In one embodiment of the present invention the particles are sized to easily pass through a 27-30 gauge needle. A characteristic of the particles produced from the biocompatible protein material is that they no longer aggregate when in the fully hydrated particulate state. Furthermore, prior studies have demonstrated that the particles do not aggregate in saline and are easily delivered through small gauge needles. The particles can be made to disassociate at very slow or fast rates in aqueous solutions. It is also noted that generally, many particle embodiments of the present invention are substantially insoluble thereby allowing them to be integrated and remodeled by the host tissue rather than excreted.

Particles of the present invention are advantageous for a variety of reasons. For example, the size and shape of the particles of the present invention provide a way to adjust the biological response of the host tissue (e.g. particles of the present invention have been found to fit and intermingle in the interstices of the host tissue, thereby enhance the bulking characteristics, biodurability or bioduration of the particles; particles also allows the material to be interdispersed or interspaced in the host tissue). Particles also provide a slower drug release matrix in comparison to gells, viscous solution etc. Furthermore, particles also provide a barrier to which most of the drug is not in direct contact with tissue and can be controllably released through a number of matrix related mechanisms (e.g. ion pairing, diffusion, enzymatic degradation, surface erosion, bulk erosion, etc.).

Embodiments of the resulting particles of the present invention utilizing any of the alternative techniques identified above, usually have as little solvent as possible while still being cohesive and possessing the desired features relevant to the particle's function, e.g., preferably a solvent content of from about 5% to about 60%, more preferably a solvent content of from about 10% to about 50% and most preferably 20% to 40%.

The particles may also be aggregated or crosslinked following formation and/or after administration (e.g. injection) to a patient by including a photoinitiator or a chemical initiator on one or more components of the particles. For example, one or more proteins (e.g. collagen) or additives (e.g. hyaluronic acid), may include a photoinitiator or chemical initiator that when activated bind the particles to each other or to a surface they come in contact with, such as tissue or a medical device. Preferably a nontoxic photoinitiator such as eosin Y photoinitiator is used. Other initiators include 2,2-imethoxy-2-phenylacetophenone and ethyl eosin. The polymerization process can be catalyzed by light or chemical in a variety of ways, including UV polymerization with a low intensity lamp emitting at about 365 nM, visible laser polymerization with an argon ion laser emitting at about 514 nM, visible illumination from a conventional endoilluminator used in vitreous surgery, and most preferably by illuminating with a lamp that emits light at a wavelength between 400-600 nM, such as, for example, a 1-kW Xe arc lamp. Illumination occurs over about 1-120 seconds, preferably less than 30 seconds. Since the heat generated is low, photopolymerization can be carried out in direct contact with cells and tissues.

The biocompatibility and tissue response to such particles has been shown to be favorable in related cardiovascular, tissue filler and drug delivery research. Also, the activity of an attached cell, such as fibroblasts, can be altered by changes in the fabrication technique (compression & cross-linking) and composition of the particles of the present invention. Additionally, cells can take on different shapes depending upon the type of particle they contact. The ability of cells to take on different shapes is indicative of their ability to respond to their environment for specialized cell functions (e.g., differentiation, proliferation).

The combined preliminary work aimed at the processing, the biocompatibility, the drug release, and the cell attachment capabilities demonstrate that the particles of the present invention can be applied as materials for numerous clinical applications including many areas of tissue filler and tissue repair, tissue regeneration, hair stimulation, bulking, medical device coating, bandages and dressings, wound healing, skin treatment and rejuvenation, biocompatible barriers and drug delivery.

The processing of the particles can be tailored for many specific applications and forms. For application to tissue and drug delivery products, particles may be produced by preparing a cohesive body that includes a base protein material including proteins such as insoluble collagen, insoluble elastin and/or albumen, and solvents, such as water, DMSO and/or glycerol. The cohesive body is then solidified utilizing one or more of the above mentioned solidification steps (e.g. heating, freezing fracturing, compression . . . ). One or more pharmacologically active agents such as those listed above may also be included in the cohesive body. The solidified cohesive body may then be processed into particles thereby producing a therapeutic device (e.g. tissue filler or drug delivery particles).

After the particles are formed using the various methods described above, they are characterized for their basic structure. First the particles may be segregated using a series of pharmaceutical drug sieves. Additional characterization of the particles will consist of verification of the shape and size of the particles using light and electron microscopy.

The particles of the present invention may be administered to a patient by a number of administration techniques know in the art. Examples of such techniques include, but are not limited to, injection, implantation, or administered via oral, as well as nasal, sublingual, intradermal, pulmonary, ocular, aural, intracranial, intravessel (i.e. intravessel walls), intranervous tissue, intramuscular, intravenous, intracardiac, transdermal, subdural, intraventricular, subcutaneous, or any other parenteral mode of delivery. Depending on the desired therapeutic effect, the particles of the present invention may be used to regenerate tissue, repair tissue, replace tissue, and deliver local and systemic therapeutic effects such as analgesia or anesthesia, or alternatively, may be used to treat specific conditions, such as skin wounds, wrinkles, internal injuries, cornea trauma, tumors or cancer sites, and other tissue specific conditions.

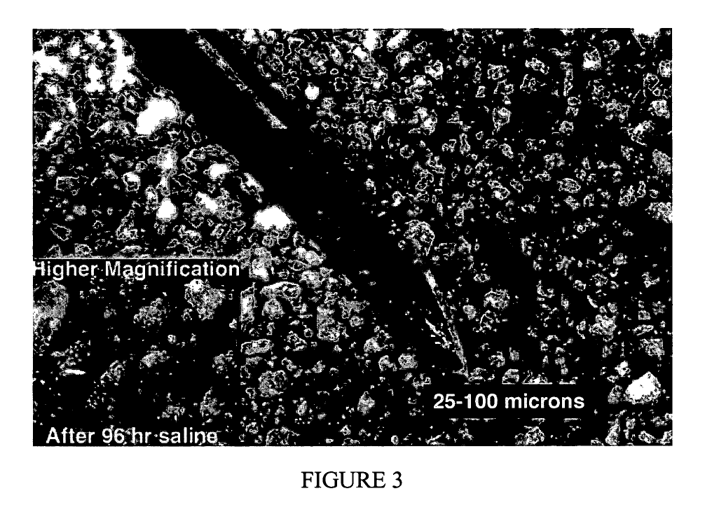

In various embodiments of the present invention, the particles may be utilized as a tissue filler or wrinkle filler by administering them subcutaneously or intradermally to the patient by a variety of administration techniques known in the art. One such administration procedure of the present invention includes the injection of the particles in a slurry or in a wetted state into the desired site by syringe. This procedure may be administered when the particles are placed in solution for delivery or are simply in a wetted state. Wetted particles generally do not have excess solvent and are flexible and/or compressible to easily fit through a needle smaller in gauge size than the actual size of the particles. Saline is a solution that may be employed to prepare the slurry or wet the particles, but any biocompatible solution may be utilized. Saline has been selected for the initial material for several reasons including its common use in medical procedures and its availability in a sterile form. However, any suitable solvent may be utilized to produce the slurry or wet the particles of the present invention. The slurry or wetted particles may be delivered in any way known in the art including delivery through a needle. Any gauge needle may be utilized to deliver the slurry containing the particles of the present invention, including but not limited to 12-30 gauge needles. FIG. 3 depicts one embodiment of a slurry of the present invention including particles in saline solution being passed through a syringe. It is noted that the particles may optionally include one or more pharmacologically active agents. However, a suitable tissue filler may also omit the inclusion of pharmacologically active agents.

Alternatively, the particles of the present invention may also be placed into position without utilizing needles. These particles are typically 10 nm-1.75 mm, preferably 500 nm-1.5 mm and more preferably 1-1000 μm. In one such a procedure the particles may be surgically implanted and packed into and/or around the injured site. For example, particles may be surgically packed into and around an injured or vacant area, such as a fractured bone or wrinkle, and subsequently sealed into position by the host tissue surrounding the injured or vacant area. The injection or implantation of biocompatible protein particles of the present invention allows for the particles to remodel with and/or resorb into the surrounding tissue or remain positioned in the injured or vacant area after it has mended or healed.

In another embodiment of the invention the particles may be administered as a hemostat, thereby dehydrating a wound site. This may be accomplished by administering the particles to a wound through a burst of air, through a dressing, sprinkling the particles, packing the particles, by a particle solution or any other means that would substantially disperse the particles uniformly over the wound site.

In yet another embodiment of the present invention the particles may be administered by a pulmonary means, nasally, orally or through the skin by devices which utilize a burst of air or spray of particles in solution, such as inhalers, nasal sprays, compressed air injectors and the like.

Additionally, the particles of the present invention may be combined with one or more excipients, carriers, coatings or adjuvants before they are administered to form a particle formulation or composition. The excipients, carriers, coatings or adjuvants preserve the singularity of each particle in each individual dose, inhibit aggregation of particles and allow for the quick or slow dispersion of the particles once administered. For example, the rapid dispersion of the particles allows the particles to disperse and possibly attach throughout the administration site. Alternatively, the particles may be combined with an excipient, carrier, coating or adjuvant formulation that slows the release of the particles thereby localizing them for a desired period of time.

Formulations or compositions suitable for use in the practice of the present invention may come in a variety of forms including, but not limited to, capsules, gels, cachets, tablets, coatings, effervescent or non-effervescent powders or tablets, powders or granules; as a solution or suspension in aqueous or non-aqueous liquid; or as an oil-in-water liquid emulsion or a water-in-oil emulsion. The compounds of the present invention may also be presented as a bolus, electuary, or paste.

Generally, formulations or compositions are prepared by uniformly mixing the particles with liquid carriers or finely divided solid carriers or both, and then if necessary shaping the product. A pharmaceutical carrier is selected on the basis of the chosen route of administration and standard pharmaceutical practice. Each carrier must be “acceptable” in the sense of being compatible with the other ingredients of the formulation and not injurious to the subject. This carrier can be a solid or liquid and the type is generally chosen based on the type of administration being used. Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, lubricants (e.g. hyaluronic acid), emulsifying agents, suspending agents, permeation enhancers, diluents, sweeteners, thickeners, and melting agents. Preferred carriers are edible oils, for example, corn or canola oils. Polyethylene glycols, e.g., PEG, are also preferred carriers. Other examples of various non-toxic, pharmaceutically acceptable, inert carriers include substances such as lactose, starch, sucrose, glucose, fructose, dextrose, methyl cellulose, magnesium stearate, carrageenan, dicalcium phosphate, calcium sulfate, mannitol, sorbitol, cyclodextrin, cyclodextrin derivatives, or the like.

Exemplary pharmaceutically acceptable carriers and excipients that may be used to formulate oral dosage forms of the present invention are described in U.S. Pat. No. 3,903,297 to Robert, issued Sep. 2, 1975, or the Handbook of Pharmaceutical Excipients, by Arthur H. Kibbe(Editor), Ainley Wade and Paul J. Weller, Amer. Pharmaceutical Assoc.; 3rd edition (Jan. 15, 2000), both of which are incorporated by reference herein in their entirety. Techniques and compositions for making dosage forms useful in the present invention are described in the following references: 7 Modern Pharmaceutics, Chapters 9 and 10 (Banker & Rhodes, Editors, 1979); Lieberman et al., Pharmaceutical Dosage Forms: Tablets (1981); and Ansel, Introduction to Pharmaceutical Dosage Forms 2nd Edition (1976).

Formulations suitable for parenteral administration include aqueous and non-aqueous formulations isotonic with the blood of the intended recipient; and aqueous and non-aqueous sterile suspensions which may include suspending systems designed to target the compound to blood components or one or more organs. The formulations may be presented in unit-dose or multi-dose sealed containers, for example, ampoules or vials. Extemporaneous injections, solutions and suspensions may be prepared from sterile powders, granules and tablets of the kind previously described. Parenteral and intravenous forms may also include minerals and other materials to make them compatible with the type of injection or delivery system chosen.

As previously suggested, the tablets, cylinders, wafers, ect. may contain suitable carriers, binders, lubricants, diluents, disintegrating agents, coloring agents, flavoring agents, flow-inducing agents, or melting agents. A tablet may be made by compression or molding the particles of the present inventon optionally with one or more additional ingredients. The compression may be performed by any device known in the art, such as a conventional pill press or any other device that forms a material by compression. Compressed tablets may be prepared by compressing the particles in a free flowing form (e.g., powder, granules) optionally mixed with a binder (e.g., gelatin, glycerin, hydroxypropylmethylcellulose, povodone, carbocol, polyvinylalcohol), lubricant, inert diluent, preservative, disintegrant (e.g., sodium starch glycolate, cross-linked carboxymethyl cellulose) surface-active or dispersing agent. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, or the like. Lubricants used in these dosage forms include hyaluronic acid, sodium oleate, sodium stearate, magnesium stearate, sodium benzoate, sodium acetate, sodium chloride, or the like. Disintegrators include, for example, starch, methyl cellulose, agar, bentonite, xanthan gum, or the like. Molded tablets may be made by molding in a suitable machine a mixture of the particles of the present invention moistened with an inert liquid diluent.

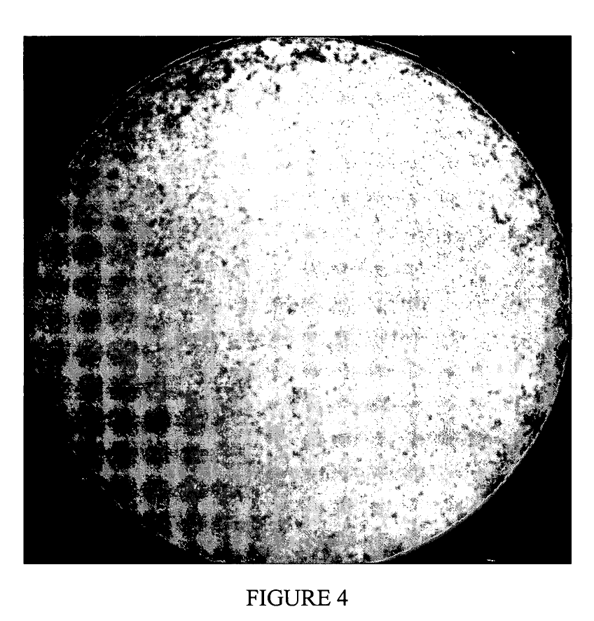

One example of a drug delivery device formed into a tablet, wafer, or cylinder may include particles prepared with one or more natural proteins, such as collagen, keratin, fibronectin, silk, silk fibroin, actin, myosin, fibrinogen, thrombin, aprotinin, elastin and/or albumen, one or more biocompatible solvents such as water, DMSO, ethanol and/or glycerol and one or more pharmacologically active agents, such as fentynal, capsaicin, ibuprofen, acetaminophen or desmopressen compressed in a compression device, such as a pill press, to produce a drug delivery device. FIG. 4 depicts one embodiment of the particles of the present invention(7 parts collagen, 2 parts elastin and 1 part heparin) compressed into a wafer form. Such a delivery device can be implanted or administered to a wound to thereby deliver the incorporated pharmacologically active agent from within the particles.

The particles or tablets, cylinders, wafers, etc. including the particles may optionally be coated or scored and may be formulated so as to provide slow- or controlled-release of the active ingredient. The coatings may be utilized to retain the particles while passing through the oral tract and into the stomach. Tablets may also optionally be provided with an enteric coating to provide release in parts of the gut other than the stomach. Additionally, the tablets may be coated on one side to act as a dissolution barrier when the opposite side is attached to an administration site.

Finally, the particles of the present invention may be included in a coating material that may be utilized to coat medical devices. For example, a polymeric coating, such as polyurethane, polytetrafluoroethylene, polyalkylmethacrylates, polyarylmethacrylates, poly(ethylene-co-vinyl acetate), or any other polymer or combination of polymers, may be homogenously combined with a plurality of particles of the present invention and applied to a medical device. The mixture of the particles in the coating material would allow for the controlled release of the contents of such particles, thereby delivering a therapeutic effect. Such coatings may be applied to any medical device known in the art including, but not limited to drug-delivering vascular stents (e.g., self-expanding stents typically made from nitinol, balloon-expanded stents typically prepared from stainless steel); other vascular devices (e.g., grafts, catheters, valves, artificial hearts, heart assist devices); implantable defibrillators; blood oxygenator devices (e.g., tubing, membranes); surgical devices (e.g., sutures, staples, anastomosis devices, vertebral disks, bone pins, suture anchors, hemostatic barriers, clamps, screws, plates, clips, vascular implants, tissue adhesives and sealants, tissue scaffolds); membranes; cell culture devices; chromatographic support materials; biosensors; shunts for hydrocephalus; wound management devices; endoscopic devices; infection control devices; orthopedic devices (e.g., for joint implants, fracture repairs); dental devices (e.g., dental implants, fracture repair devices), urological devices (e.g., penile, sphincter, urethral, bladder and renal devices, and catheters); colostomy bag attachment devices; ophthalmic devices (e.g. intraocular coils/screws); glaucoma drain shunts; synthetic prostheses (e.g., breast); intraocular lenses; respiratory, peripheral cardiovascular, spinal, neurological, dental, ear/nose/throat (e.g., ear drainage tubes); renal devices; and dialysis (e.g., tubing, membranes, grafts), urinary catheters, intravenous catheters, small diameter grafts, vascular grafts, artificial lung catheters, atrial septal defect closures, electro-stimulation leads for cardiac rhythm management (e.g., pacer leads), glucose sensors (long-term and short-term), degradable coronary stents (e.g., degradable, non-degradable, peripheral), blood pressure and stent graft catheters, birth control devices, BHP and prostate cancer implants, bone repair/augmentation devices, breast implants, cartilage repair devices, dental implants, implanted drug infusion tubes, intravitreal drug delivery devices, nerve regeneration conduits, oncological implants, electrostimulation leads, pain management implants, spinal/orthopedic repair devices, wound dressings, embolic protection filters, abdominal aortic aneurysm grafts, heart valves (e.g., mechanical, polymeric, tissue, percutaneous, carbon, sewing cuff), valve annuloplasty devices, mitral valve repair devices, vascular intervention devices, left ventricle assist devices, neuro aneurysm treatment coils, neurological catheters, left atrial appendage filters, hemodialysis devices, catheter cuff, anastomotic closures, vascular access catheters, cardiac sensors, uterine bleeding patches, urological catheters/stents/implants, in vitro diagnostics, aneurysm exclusion devices, and neuropatches.

Examples of other suitable devices include, but are not limited to, vena cava filters, urinary dialators, endoscopic surgical tissue extractors, atherectomy catheters, clot extraction catheters, PTA catheters, PTCA catheters, stylets (vascular and non-vascular), coronary guidewires, drug infusion catheters, esophageal stents, circulatory support systems, angiographic catheters, transition sheaths and dialators, coronary and peripheral guidewires, hemodialysis catheters, neurovascular balloon catheters, tympanostomy vent tubes, cerebro-spinal fluid shunts, defibrillator leads, percutaneous closure devices, drainage tubes, thoracic cavity suction drainage catheters, electrophysiology catheters, stroke therapy catheters, abscess drainage catheters, biliary drainage products, dialysis catheters, central venous access catheters, and parental feeding catheters.

Other examples of medical devices suitable for the present invention include, but are not limited to implantable vascular access ports, blood storage bags, blood tubing, central venous catheters, arterial catheters, vascular grafts, intraaortic balloon pumps, cardiovascular sutures, total artificial hearts and ventricular assist pumps, extracorporeal devices such as blood oxygenators, blood filters, hemodialysis units, hemoperfusion units, plasmapheresis units, hybrid artificial organs such as pancreas or liver and artificial lungs, as well as filters adapted for deployment in a blood vessel in order to trap emboli (also known as “distal protection devices”). It is noted that in other embodiments of the present invention, the particles of the present invention may also be adhered to the medical device by means other that coatings materials, such as adhesives or compression.

In yet other embodiments of the present invention, the particles may be compressed or adhered to other medical devices such as stents or pacemakers to form a biocompatible coating. In various embodiments of the present invention biocompatible surfaces can be created by adhereing the particles of the present invention to a polymeric material to form a biocompatible surface material.

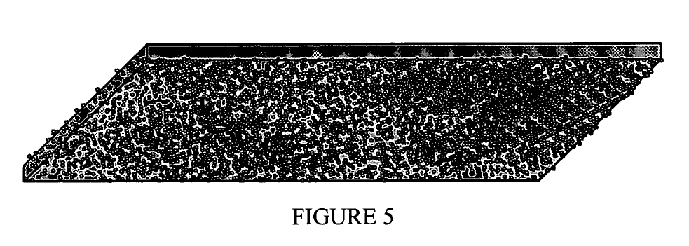

FIG. 5 depicts another embodiment of a the present invention in the form of a biocompatible surface material. The biocompatible surface material generally comprises a polymeric base, which binds an outer surface of biocompatible particles. In various embodiments of the present invention the biocompatible particles are homogenously distributed over and at least partially embedded in the surface of the polymeric material thereby providing an enhanced biocompatible surface. The polymeric materials with biocompatible surfaces of the present invention have enhanced biocompatible attributes, which include their capacity to decrease thrombogenicity, reduce an inflammatory response, to allow direct cell integration, to deliver therapeutic agents, to allow regeneration of host tissue into the graft and/or to allow other graft materials to adhere to their surface.

The polymeric base may be produced utilizing any binding polymeric material. However, a biostable and/or bioabsorbable polymeric material may provide an optimum polymeric base. For example, biostable and/or bioabsorbable polymers that could be used in the present invention include, but are not limited to poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, and polyesters could be used and other polymers could also be used if they can be dissolved in a solvent and coated on a surface, such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, polyvinyl pyrrolidone, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The process of the present invention for preparing the polymeric material including a biocompatible surface comprises applying a polymeric base to a surface. The surface may be any surface capable of being coated, such as a table top, glass substrate, medical devices such as pacemakers or stents, leads, antennas or any other surface that can support a coating. Such surfaces can represent the final coated surface or can serve as a temporary surface from which the coating can be peeled off to provide a separate polymer film. The polymeric material may be applied to the surface by any suitable application method known in the art, such as spray coating, dip coating, knife coating or the like. Generally, the polymeric material is solvent cast onto a surface. The solution of polymeric material is initially in a nonpolymerized state before application to a surface, such as in a liquid form of individual monomers or a semipolymerized state.

Once the polymeric solution is applied to the surface, biocompatible particles are next administered to the polymeric solution and the polymeric solution is allowed to dry, cure and/or polymerize thereby binding the biocompatible particles to the polymeric material to form a polymeric material with a biocompatible surface. Any suitable particle administration methods know in the art may be utilized to administer the particles to the polymer coated surface. For example, the particles may be administered to the surface by press rolling the polymer coated surface in the particles, spraying the particles onto the polymer, sieving the particles onto the polymer, shaking the particles onto the polymer, blowing the particles onto the polymer or by any other administration means. Finally, the biocompatible particles may be exposed on all surfaces of the polymeric material by lifting the polymeric material from the surface and cutting, scraping or abrading the side of the material that was adjacent to the surface. Such action removes the polymeric material and thereby exposes the biocompatible particles.

The polymeric materials with biocompatible surfaces may be utilized for various medical applications including, but not limited to, drug delivery devices for the controlled release of pharmacologically active agents including drug delivery patches, encapsulated or coated stent devices, vessels, tubular grafts, vascular grafts, wound healing devices including protein matrix suture material and meshes, skin/bone/tissue grafts, adhesion prevention barriers, cell scaffolding, medical device coatings/films and other biocompatible implants.

One such medical application includes vessels and tubular grafts. In one embodiment of the present invention, a vessel or tubular graft may be produced by preparing sheets of the polymeric material with biocompatible surfaces and adjoining two ends of the sheet to form a tube. The material may be adjoined by any suitable means, including but not limited to sutures, adhesives, pressure fitting, heat, ultrasonic welding, solvent welding and crosslinking. Alternatively, a vessel or tubular graft may be produced by preparing the polymeric material with biocompatible surfaces on a cylindrical surface and removing the cylinder once the material has polymerized to a state wherein the form is determined. Finally, the vessels prepared according to the present invention may include biocompatible surfaces on the interior and/or exterior of the vessel. A vessel including biocompatible interior and exterior surfaces may be prepared by either removing the polymeric material from the surface opposite the surface wherein particles were administered or by utilizing a multilayered vessel including a vessel with a biocompatible interior inserted and adhered to a larger vessel with a biocompatible exterior. It is noted that vessels may be produced wherein endothelial cells are grown on the inside of tube and smooth muscle cells on the outside of the tube.

Another medical application embodiment of the present invention include wound healing devices that utilize the polymeric material with biocompatible surfaces. The wound healing devices may be configured by forming the particle coated polymers of the present invention into any shape and size to accommodate the wound being treated. Moreover, the wound healing devices of the present invention may be produced in whatever shape and size is necessary to provide optimum treatment to the wound. These devices can be produced in the forms that include, but are not limited to, plugs, meshes, strips, sutures, or any other form able to accommodate and assist in the repair of a wound. The damaged portions of the patient that may be treated with a devices made of the particles of the present invention include, but are not limited to, skin, tissue (nerve, muscle, cartilage, brain, spinal cord, heart, lung, etc.) and bone. Moreover, the particles of the present invention, with or without the polymeric base, may be formed into various wound healing devices including, but are not limited to, dental plugs and inserts, skin dressings and bandages, bone inserts, tissue plugs and inserts, vertebrae, vertebral discs, joints (e.g., finger, toe, knee, hip, elbow, wrist,), tissue plugs to close off airway, (e.g., bronchial airway from resected tissue site), other similar devices administered to assist in the treatment repair and remodeling of the damaged tissue and/or bone.

It is also possible to extend delivery of chemicals or drugs using a polymeric material with biocompatible surfaces as previously described as a patch delivery system. In this example the particles of the biocompatible surface would include a dosage of the chemical or pharmaceutically active component. An adhesive or other adhering means may be applied to the outer edges of the polymeric material to hold the patch in position during the delivery of the chemical or pharmaceutically active component. By administering such a patch delivery system, the delivery of chemicals and/or pharmaceuticals could be systematically and/or locally administered until the desired amount of chemicals and/or pharmaceuticals were applied.

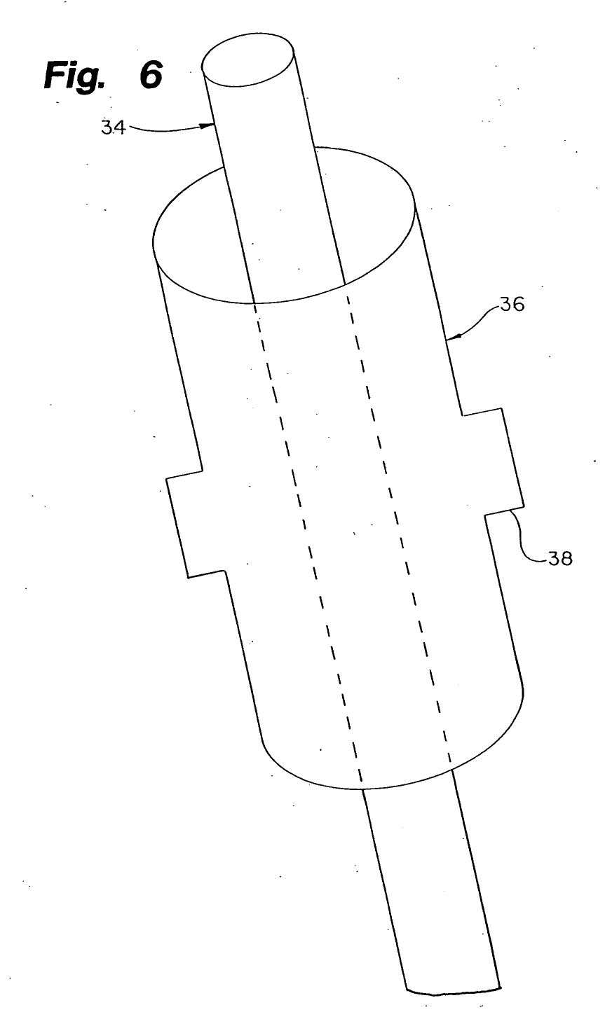

The polymeric material with biocompatible surfaces of the present invention may also be utilized as port seals for protrusion devices entering and or exiting the patient. FIG. 6 depicts one embodiment of a protrusion device 34 that includes a port seal 36 comprising the polymeric material of the present invention. The port seal 26 may be included around the point of insertion of a protrusion device, such as an electrical lead, drug administration needle, drainage tubes or a catheter. Generally, the port seal 36 surrounds the protrusion device 34 and insulates it from the host tissue. One or more tabs 38 may optionally be included on the port seal 36 to assist in the retention of the protrusion device and further seal the opening in the patients skin. The tabs 38 may be inserted under the skin or may remain on the outside of the patient's skin. Also, the biocompatible seal comprising the protein matrix material of the present invention provides stability, reduces the seeping of bodily fluid from around the protrusion and reduces or prevents inflammation caused by the protrusion device. Furthermore, the port seal may include pharmacologically active agents that may be produced to deliver anti-bacterial, analgesic, anti-inflammatory and/or other beneficial pharmacologically active agents.

Other embodiments of the present invention include wound-healing devices configured and produced as polymeric material biological fasteners, such as threads, sutures and woven sheets. Threads and sutures comprising various embodiments of the polymeric material provide a biocompatible fastening and suturing function for temporarily treating and sealing an open wound. Additionally, the biological fasteners may include pharmacologically active agents that may assist in the healing and remodeling of the tissue within and around the wound.

One method of preparing the biocompatible biological fasteners is to manufacture sheets of polymeric material with biocompatible surfaces. Once the sheets of protein matrix material are prepared each sheet may cut into strips, threads or other shapes to form sutures, threads and other biological fasteners (e.g., hemostats). The sheets may be cut using cutting techniques known in the art.

Additional embodiments of medical applications that include the particles, with or without the polymeric base, include but are not limited to wound inserts, wound plugs, wound implants, wound adhesives, dental inserts, dental plugs, dental implants, dental adhesives, and other devices utilized for dental applications. Wounds and dental complications, such as dry socket, present within the interior of the mouth are generally slow to heal, are painful and/or are susceptible to bacterial and other forms of infection. The particles, dental inserts or implants of the present invention may be utilized to remedy such problems since they are biocompatible with the surrounding host tissue and may be manufactured to release appropriate pharmacologically active agents that may assist in healing, relieve pain and/or reduce bacterial attack of the damaged region. Furthermore, the particles, dental plugs, inserts or implants of the present invention generally include one or more biocompatible purified protein materials and one or more biocompatible solvents that may be incorporated into and remodeled by the surrounding tissue, thereby hastening the healing of the damaged region and/or returning the damaged region to its original state. For example, particles, dental plugs or implants may be administered by sprinkling, packing, implanting, inserting or applying by any other administration means to open wounds on the body. These particles or devices made from the particles may be beneficial in treating wounds within the mouth region of the patient, such as mucositis, or for treating wounds following tooth extraction, oral surgery or any other type of injury to the interior of the mouth. Alternatively, the wound may also be treated by packing the wound or covering the wound with particles formed into a desired shape for applying to a wound by molding the particles. One method for forming particles into a desired shape is by compression. Application of such particle devices assist in the healing and regeneration of the damaged region.

### EXAMPLE I

### Collagen Modified Polyurethane Surface

Bovine fibrous collagen (1.715 g) was mixed with elastin (0.457 g) and heparin (0.114 g) in a two-syringe mixing system with the addition of 5 ml of distilled water and 3 ml of phosphate buffered saline (pH 7.4). When the mixture appeared uniform, the resulting material was dehydrated at 30° C. until 60% of the added water was removed. This paste (B-stage) was stored at 42° F. overnight. The B-stage was made into smaller pieces suitable for use in a single ball grinding device held at liquid nitrogen temperature. This grinding resulted in a particulate material which could be used as the surface treatment for a polyurethane film, which was prepared by casting a solution of Chronoflex-AR from DMAC (22% solids) and partially drying the film at 65° C. until the surface reached a semi-solid, sticky state. The collagenous particulate material was then uniformly added to this surface using a shaker device and the resulting composition dried overnight at 65° C. The final modified polyurethane surface was then hydrated and the excess particulate material removed. This modified polyurethane film, having a collagen/elastin/heparin embedded surface, was then ready for fabrication into the appropriate body-contacting surface, such as a vascular graft.

### EXAMPLE II

Bovine fibrous collagen (1.715 g) was mixed with elastin (0.457 g) and heparin (0.114 g) in a two-syringe mixing system with the addition of 5 ml of distilled water and 3 ml of phosphate buffered saline (pH 7.4). When the mixture appeared uniform, it was spread on a flat surface and dehydrated overnight at 40° C. to yield a solid. This solid was broken into pieces and ground at liquid nitrogen temperature to yield particles.

### EXAMPLE III

### Cross-Linking of Collagen/Elastin/Heparin Cohesive Body

The glutaraldehyde treatment of a cohesive body including collagen, elastin and heparin at a 7/2/1 ratio is as follows: add 0.2 ml of 50% aqueous glutaraldehyde to 100 ml of distilled water. To the stirred solution (magnet stir bar) add fully-hydrated cohesive body pieces (no more than 14 grams has been used at this point) and stir slowly (just enough to move the cohesive body pieces) for 2 hours at ambient temperature. The pieces are rinsed three times with fresh distilled water. Next 100 ml of water is added to the beaker with cohesive body pieces and approximately 0.13 g of glycine and 0.13 g of glutamine is added to the beaker and stirred slowly for 30 minutes. Next, the cohesive body pieces are rinsed 3 times with fresh water. The crosslinked cohesive body pieces are then removed from the beaker and placed on a glass plate or weighing dish and dried at 50° C. for approximately 48 hours.

### EXAMPLE IV

### Particle Processing

One particle formation process is as follows: The crosslinked cohesive body of Example III is ground in a reciprocating grinding system until all ground material passes through a 150 micron sieve. The final ground particles are added to a beaker containing approximately 30-50 mls of PBS stirred sufficiently to fully disperse the particles—no clumping is allowed. The dispersed particles are allowed to settle overnight in the refrigerator. The supernatant is decanted or pipetted off and the suspended particles are “dewatered” by any of several methods (wicking, centrifugation, compression between absorbant materials). The dewatered particles are next added to at least a 6 ml syringe at the plunger end and then injected into 1 ml syringes through a metal syringe connector. The final 1 ml syringe is then sterilized with approximately 60 Krads of gamma radiation and stored in the refrigerator ready for use. The particles are suitable for injection through a 30 gauge or larger bore needle.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations, which fall within the spirit and broad scope of the invention.

1. A biocompatible protein particulate material comprising a plurality of protein particles, said protein particles including one or more biocompatible purified proteins, combined with one or more biocompatible solvents to form a cohesive body that is subsequently solidified and processed into particles.

2. The biocompatible protein particulate material of claim 1 wherein the particles have a size approximately equal to or less than 2 mm.

3. The biocompatible protein particulate material of claim 1 wherein the biocompatible proteins are selected from the group consisting of elastin, collagen, albumin, ovalbumen, keratin, laminin, fibronectin, silk, silk fibroin, actin, myosin, fibrinogen, thrombin, aprotinin, antithrombin III, elastinlike blocks, silklike blocks, collagenlike blocks, lamininlike blocks, fibronectinlike blocks and silklike, elastinlike blocks, collagen-heparin, collagen-elastin-heparin and collagen-chondroiten.

4. The biocompatible protein particulate material of claim 1 wherein the biocompatible solvent is selected from the group consisting of water, dimethyl sulfoxide (DMSO), biocompatible alcohols, biocompatible acids, oils and biocompatible glycols.

5. The biocompatible protein particulate material of claim 1 further including one or more pharmacologically active agents wherein the one or more pharmacologically active agents are selected from the group consisting of analgesics, anesthetics, antipsychotic agents, angiogenic growth factors, bone mending biochemicals, steroids, antisteroids, corticosteroids, antiglacoma agents, antialcohol agents, anti-coagulant agents, genetic material, antithrombolytic agents, anticancer agents, anti-Parkinson agents, antiepileptic agents, permeation enhancers, anti-inflammatory agents, anticonception agents, enzymes agents, cells, growth factors, antiviral agents, antibacterial agents, antifungal agents, hypoglycemic agents, antihistamine agents, chemoattractants, neutraceuticals, antiobesity, smoking cessation agents, obstetric agents and antiasmatic agents.

6. The biocompatible protein particulate material of claim 5 wherein the pharmacologically active agents are analgesics, antiinflammatories, anti-coagulant agents, anesthetics or neurotoxins.

7. The biocompatible protein particulate material of claim 1 further comprising one or more biocompatible additives.

8. The biocompatible protein particulate material of claim 7 wherein the one or more biocompatible additives are selected from the group consisting of epoxies, polyesters, acrylics, nylons, silicones, polyanhydride, polyurethane, polycarbonate, poly(tetrafluoroethylene), polycaprolactone, polyethylene oxide, polyethylene glycol, poly(vinyl chloride), polylactic acid, polyglycolic acid, polypropylene oxide, poly(akylene)glycol, polyoxyethylene, sebacic acid, polyvinyl alcohol, 2-hydroxyethyl methacrylate, polymethyl methacrylate, 1,3-bis(carboxyphenoxy)propane, lipids, phosphatidylcholine, triglycerides, humectants, polyhydroxybutyrate, polyhydroxyvalerate, poly(ethylene oxide), poly ortho esters, poly (amino acids), polycyanoacrylates, polyphophazenes, polysulfone, polyamine, poly (amido amines), fibrin, graphite, flexible fluoropolymer, isobutyl-based, isopropyl styrene, vinyl pyrrolidone, cellulose acetate dibutyrate, silicone rubber, and copolymers or combinations of these.

9. The biocompatible protein particulate material of claim 1 wherein all or a portion of the particles are crosslinked with one or more crosslinking agents.

10. The biocompatible protein particulate material of claim 9 wherein the one or more crosslinking agents are selected from the group consisting of glutaraldehyde, formaldehyde, p-Azidobenzolyl Hydazide, N-5-Azido 2-nitrobenzoyloxysuccinimide, 1,4-butandiol diglycidylether, N-Succinimidyl 6-[4′azido-2′nitro-phenylamino]hexanoate and 4-[p-Azidosalicylamido]butylamine.

11. The biocompatible protein particulate material of claim 1 wherein the particles have a solvent content of approximately 10% to about 60%.

12. The biocompatible protein particulate material of claim 11 wherein the particles have a solvent content of approximately 30% to about 50%.

13. A method of treating an injured or vacant portion of a patient's body comprising:
administering a plurality of protein particles to the injured or vacant portion of the patient's body, said protein particles including one or more biocompatible purified proteins interacting with one or more biocompatible solvents.

14. The method of treating an injured or vacant portion of a patient's body of claim 13 wherein the particles have a size of approximately 1 μm to 1000 μm.

15. The method of treating an injured or vacant portion of a patient's body of claim 13 wherein the biocompatible proteins are selected from the group consisting of elastin, collagen, albumin, keratin, laminin, fibronectin, silk, silk fibroin, actin, myosin, fibrinogen, thrombin, aprotinin, antithrombin III, elastinlike blocks, silklike blocks, collagenlike blocks, lamininlike blocks, fibronectinlike blocks and silklike, elastinlike blocks, collagen-heparin, collagen-elastin-heparin and collagen-chondroiten.

16. The method of treating an injured or vacant portion of a patient's body of claim 13 wherein the biocompatible solvent is selected from the group consisting of water, dimethyl sulfoxide (DMSO), biocompatible alcohols, biocompatible acids, oils and biocompatible glycols.

17. The method of treating an injured or vacant portion of a patient's body of claim 13 wherein the tissue filler further includes one or more pharmacologically active agents selected from the group consisting of analgesics, anesthetics, antipsychotic agents, angiogenic growth factors, bone mending biochemicals, steroids, antisteroids, corticosteroids, antiglacoma agents, antialcohol agents, anti-coagulants agents, genetic material, antithrombolytic agents, anticancer agents, anti-Parkinson agents, antiepileptic agents, permeation enhancers, anti-inflammatory agents, anticonception agents, enzymes agents, cells, growth factors, antiviral agents, antibacterial agents, antifingal agents, hypoglycemic agents, antihistamine agents, chemoattractants, neutraceuticals, antiobesity, smoking cessation agents, obstetric agents and antiasmatic agents.

18. The method of treating an injured or vacant portion of a patient's body of claim 17 wherein the pharmacologically active agent is selected from anesthetics, analgesics, anti-coagulant agents or neurotoxins.

19. The method of treating an injured or vacant portion of a patient's body of claim 13 further comprising one or more biocompatible additives.

20. The method of treating an injured or vacant portion of a patient's body of claim 19 wherein the one or more biocompatible additives are selected from the group consisting of epoxies, polyesters, acrylics, nylons, silicones, polyanhydride, polyurethane, polycarbonate, poly(tetrafluoroethylene), polycaprolactone, polyethylene oxide, polyethylene glycol, poly(vinyl chloride), polylactic acid, polyglycolic acid, polypropylene oxide, poly(akylene)glycol, polyoxyethylene, sebacic acid, polyvinyl alcohol, 2-hydroxyethyl methacrylate, polymethyl methacrylate, 1,3-bis(carboxyphenoxy)propane, lipids, phosphatidylcholine, triglycerides, humectants, polyhydroxybutyrate, polyhydroxyvalerate, poly(ethylene oxide), poly ortho esters, poly (amino acids), polycyanoacrylates, polyphophazenes, polysulfone, polyamine, poly (amido amines), fibrin, graphite, flexible fluoropolymer, isobutyl-based, isopropyl styrene, vinyl pyrrolidone, cellulose acetate dibutyrate, silicone rubber, and copolymers or combinations of these.

21. The method of treating an injured or vacant portion of a patient's body of claim 13 wherein all or a portion of the particles are crosslinked with one or more crosslinking agents.

22. The method of treating an injured or vacant portion of a patient's body of claim 21 wherein the one or more crosslinking agents are selected from the group consisting of glutaraldehyde, formaldehyde, p-Azidobenzolyl Hydazide, N-5-Azido 2-nitrobenzoyloxysuccinimide, 1,4-butandiol diglycidylether, N-Succinimidyl 6-[4′azido-2′nitro-phenylamino]hexanoate and 4-[p-Azidosalicylamido]butylamine.

23. The method of treating an injured or vacant portion of a patient's body of claim 13 wherein the particles have a solvent content of approximately 10% to about 60%.

24. The method of treating an injured or vacant portion of a patient's body of claim 23 wherein the particles have a solvent content of approximately 30% to about 50%.

25. The method of treating an injured or vacant portion of a patient's body of claim 13 wherein the injured or vacant portion is a wrinkle, bone fracture, skin wound, buccal cavity or gum injury, surgical wound or mucosal tissue wound.

26. The method of treating an injured or vacant portion of a patient's body of claim 13 wherein the particles are further compressed to form a tablet, wafer, cylinder or sheet.

27. A drug delivery device comprising a plurality of protein particles, said protein particles including one or more biocompatible purified proteins interacting with one or more biocompatible solvents and including one or more pharmacologically active agents.

28. The drug delivery device of claim 27 wherein the particles have a size of approximately 1 μm to 1000 μm.

29. The drug delivery device of claim 27 wherein the biocompatible proteins are selected from the group consisting of elastin, collagen, albumin, keratin, laminin, fibronectin, silk, silk fibroin, actin, myosin, fibrinogen, thrombin, aprotinin, antithrombin III, elastinlike blocks, silklike blocks, collagenlike blocks, lamininlike blocks, fibronectinlike blocks and silklike, elastinlike blocks, collagen-heparin, collagen-elastin-heparin and collagen-chondroiten.

30. The drug delivery device of claim 27 wherein the biocompatible solvent is selected from the group consisting of water, dimethyl sulfoxide (DMSO), biocompatible alcohols, biocompatible acids, oils and biocompatible glycols.

31. The drug delivery device of claim 27 wherein the one or more pharmacologically active agents are selected from the group consisting of analgesics, anesthetics, antipsychotic agents, angiogenic growth factors, bone mending biochemicals, steroids, antisteroids, corticosteroids, antiglacoma agents, antialcohol agents, anti-coagulants agents, genetic material, antithrombolytic agents, anticancer agents, anti-Parkinson agents, antiepileptic agents, permeation enhancers, anti-inflammatory agents, anticonception agents, enzymes agents, cells, growth factors, antiviral agents, antibacterial agents, antifungal agents, hypoglycemic agents, antihistamine agents, chemoattractants, neutraceuticals, antiobesity, smoking cessation agents, obstetric agents and antiasmatic agents.

32. The drug delivery device of claim 31 wherein the pharmacologically active agents are selected from analgesics, anesthetics, antibacterial agents, antifungal agents, antiinflammatories, or antidiuretics.

33. The drug delivery device of claim 27 further comprising one or more biocompatible additives.

34. The drug delivery device of claim 33 wherein the one or more biocompatible additives are selected from the group consisting of epoxies, polyesters, acrylics, nylons, silicones, polyanhydride, polyurethane, polycarbonate, poly(tetrafluoroethylene), polycaprolactone, polyethylene oxide, polyethylene glycol, poly(vinyl chloride), polylactic acid, polyglycolic acid, polypropylene oxide, poly(akylene)glycol, polyoxyethylene, sebacic acid, polyvinyl alcohol, 2-hydroxyethyl methacrylate, polymethyl methacrylate, 1,3-bis(carboxyphenoxy)propane, lipids, humectants, phosphatidylcholine, triglycerides, polyhydroxybutyrate, polyhydroxyvalerate, poly(ethylene oxide), poly ortho esters, poly (amino acids), polycyanoacrylates, polyphophazenes, polysulfone, polyamine, poly (amido amines), fibrin, graphite, flexible fluoropolymer, isobutyl-based, isopropyl styrene, vinyl pyrrolidone, cellulose acetate dibutyrate, silicone rubber, and copolymers or combinations of these.

35. The drug delivery device of claim 27 wherein all or a portion of the particles are crosslinked with one or more crosslinking agents.

36. The drug delivery device of claim 35 wherein the one or more crosslinking agents are selected from the group consisting of glutaraldehyde, formaldehyde, p-Azidobenzolyl Hydazide, 1,4-butandiol diglycidylether, N-5-Azido 2-nitrobenzoyloxysuccinimide, N-Succinimidyl 6-[4′azido-2′nitro-phenylamino]hexanoate and 4-[p-Azidosalicylamido]butylamine.

37. The drug delivery device of claim 27 wherein the particles have a solvent content of approximately 10% to about 60%.

38. The drug delivery device of claim 37 wherein the particles have a solvent content of approximately 30% to about 50%

39. The drug delivery device of claim 27 wherein the drug delivery device includes one or more excipients, carriers, adjuvants or a combination thereof.

40. The drug delivery device of claim 27 wherein the particles are further compressed to form of a tablet, wafer, cylinder or sheet.

41. A method of making a biocompatible protein particulate material comprising:
(a) preparing a coatable composition including the one or more biocompatible purified protein materials and the one or more biocompatible solvents;
(b) coating the composition to form a film;
(c) partially drying the coated film until the coated film can be formed into a cohesive body;
(d) forming said cohesive body;
(e) processing the cohesive body to form a plurality of biocompatible protein particles.

42. The method of making a biocompatible protein particulate material of claim 41 further including solidifying the cohesive body before processing into particles.

43. The method of making a biocompatible protein particulate material of claim 42 wherein the cohesive body is solidified by heating, freeze fracture techniques, freeze drying or vacuum drying.

44. The method of making a biocompatible protein particulate material of claim 41 wherein the particles have a size of approximately 1 μm to 1000 μm.

45. The method of making a biocompatible protein particulate material of claim 41 wherein the biocompatible purified proteins are selected from the group consisting of elastin, collagen, albumin, keratin, laminin, fibronectin, silk, silk fibroin, actin, myosin, fibrinogen, thrombin, aprotinin, antithrombin III, elastinlike blocks, silklike blocks, collagenlike blocks, lamininlike blocks, fibronectinlike blocks and silklike, elastinlike blocks, collagen-heparin, collagen-elastin-heparin and collagen-chondroiten.

46. The method of making a biocompatible protein particulate material of claim 41 wherein the biocompatible solvent is selected from the group consisting of water, dimethyl sulfoxide (DMSO), biocompatible alcohols, biocompatible acids, oils and biocompatible glycols.

47. The method of making a biocompatible protein particulate material of claim 41 wherein the particles further include one or more pharmacologically active agents selected from the group consisting of analgesics, anesthetics, antipsychotic agents, angiogenic growth factors, bone mending biochemicals, steroids, antisteroids, corticosteroids, antiglacoma agents, antialcohol agents, anti-coagulants agents, genetic material, antithrombolytic agents, anticancer agents, anti-Parkinson agents, antiepileptic agents, anti-inflammatory agents, anticonception agents, enzymes agents, cells, growth factors, antiviral agents, antibacterial agents, antifungal agents, hypoglycemic agents, antihistamine agents, chemoattractants, neutraceuticals, antiobesity, smoking cessation agents, obstetric agents and antiasmatic agents.

48. The method of making a biocompatible protein particulate material of claim 41 wherein the particles further include one or more biocompatible additives selected from the group consisting of epoxies, polyesters, acrylics, nylons, silicones, polyanhydride, polyurethane, polycarbonate, poly(tetrafluoroethylene), polycaprolactone, polyethylene oxide, polyethylene glycol, poly(vinyl chloride), polylactic acid, polyglycolic acid, polypropylene oxide, poly(akylene)glycol, polyoxyethylene, sebacic acid, polyvinyl alcohol, 2-hydroxyethyl methacrylate, polymethyl methacrylate, 1,3-bis(carboxyphenoxy)propane, lipids, phosphatidylcholine, triglycerides, humectants, polyhydroxybutyrate, polyhydroxyvalerate, poly(ethylene oxide), poly ortho esters, poly (amino acids), polycyanoacrylates, polyphophazenes, polysulfone, polyamine, poly (amido amines), fibrin, graphite, flexible fluoropolymer, isobutyl-based, isopropyl styrene, vinyl pyrrolidone, cellulose acetate dibutyrate, silicone rubber, and copolymers or combinations of these.

49. The method of making a biocompatible protein particulate material of claim 41 wherein all or a portion of the particles are crosslinked with one or more crosslinking agents.

50. The method of making a biocompatible protein particulate material of claim 49 wherein the one or more crosslinking agents are selected from the group consisting of glutaraldehyde, formaldehyde, p-Azidobenzolyl Hydazide, N-5-Azido 2-nitrobenzoyloxysuccinimide, 1,4-butandiol diglycidylether, N-Succinimidyl 6-[4′azido-2′nitro-phenylamino]hexanoate and 4-[p-Azidosalicylamido]butylamine.

51. A polymeric material with a biocompatible particulate surface comprising a polymeric base layer integrally adjoined and exposing on at least one surface area of the material a plurality of particles comprising one or more biocompatible purified proteins interacting with one or more biocompatible solvents.

52. The polymeric material with a biocompatible particulate surface of claim 51 wherein the polymeric base layer includes one or more polymers selected from the group consisting of poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes, fibrin, fibrinogen, cellulose, starch, collagen, hyaluronic acid, polyurethanes, silicones, polyesters, polyolefins, polyisobutylene, ethylene-alphaolefin copolymers, acrylic polymers and copolymers, vinyl halide polymers and copolymers, polyvinyl chloride, polyvinyl ethers, polyvinyl methyl ether, polyvinylidene halides, polyvinylidene fluoride, polyvinylidene chloride, polyacrylonitrile, polyvinyl ketones, polyvinyl aromatics, polystyrene, polyvinyl esters, polyvinyl acetate, olefins, ethylene-methyl methacrylate polymers, polyvinyl pyrrolidone, acrylonitrile-styrene polymers, ABS resins, ethylene-vinyl acetate polymers, polyamides, Nylon 66, polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose.

53. The polymeric material with a biocompatible particulate surface of claim 51 wherein the particles have a size of approximately 1 μm to 1000 μm.

54. The polymeric material with a biocompatible particulate surface of claim 51 wherein the biocompatible proteins are selected from the group consisting of elastin, collagen, albumin, keratin, fibronectin, silk, silk fibroin, actin, myosin, fibrinogen, thrombin, aprotinin, antithrombin III, elastinlike blocks, silklike blocks, collagenlike blocks, lamininlike blocks, fibronectinlike blocks and silklike, elastinlike blocks, collagen-heparin, collagen-elastin-heparin and collagen-chondroiten.

55. The polymeric material with a biocompatible particulate surface of claim 51 wherein the biocompatible solvent is selected from the group consisting of water, dimethyl sulfoxide (DMSO), biocompatible alcohols, biocompatible acids, oils and biocompatible glycols.

56. The polymeric material with a biocompatible particulate surface of claim 51 wherein the particles further include one or more pharmacologically active agents selected from the group consisting of analgesics, anesthetics, antipsychotic agents, angiogenic growth factors, bone mending biochemicals, steroids, antisteroids, corticosteroids, antiglacoma agents, antialcohol agents, anti-coagulants agents, genetic material, antithrombolytic agents, anticancer agents, anti-Parkinson agents, antiepileptic agents, permeation enhancers, anti-inflammatory agents, anticonception agents, enzymes agents, cells, growth factors, antiviral agents, antibacterial agents, antifungal agents, hypoglycemic agents, antihistamine agents, chemoattractants, neutraceuticals, antiobesity, smoking cessation agents, obstetric agents and antiasmatic agents.

57. The polymeric material with a biocompatible particulate surface of claim 51 further comprising one or more biocompatible additives selected from the group consisting of epoxies, polyesters, acrylics, nylons, silicones, polyanhydride, polyurethane, polycarbonate, poly(tetrafluoroethylene), polycaprolactone, polyethylene oxide, polyethylene glycol, poly(vinyl chloride), polylactic acid, polyglycolic acid, polypropylene oxide, poly(akylene)glycol, polyoxyethylene, sebacic acid, polyvinyl alcohol, 2-hydroxyethyl methacrylate, polymethyl methacrylate, 1,3-bis(carboxyphenoxy)propane, lipids, phosphatidylcholine, triglycerides, humectants, polyhydroxybutyrate, polyhydroxyvalerate, poly(ethylene oxide), poly ortho esters, poly (amino acids), polycyanoacrylates, polyphophazenes, polysulfone, polyamine, poly (amido amines), fibrin, graphite, flexible fluoropolymer, isobutyl-based, isopropyl styrene, vinyl pyrrolidone, cellulose acetate dibutyrate, silicone rubber, and copolymers or combinations of these.

58. The polymeric material with a biocompatible particulate surface of claim 51 wherein all or a portion of the particles are crosslinked with one or more crosslinking agents.

59. The polymeric material with a biocompatible particulate surface of claim 58 wherein the one or more crosslinking agents are selected from the group consisting of glutaraldehyde, formaldehyde, p-Azidobenzolyl Hydazide, 1,4-butandiol diglycidylether, N-5-Azido 2-nitrobenzoyloxysuccinimide, N-Succinimidyl 6-[4′azido-2′nitro-phenylamino]hexanoate and 4-[p-Azidosalicylamido]butylamine.

60. A method of making a polymeric material with a biocompatible particulate surface comprising:
(a) applying one or more polymeric materials to a surface to form a polymeric base;
(b) administering one or more biocompatible particles to the polymeric base before the polymeric materials completely polymerize thereby embedding the particles partially into the surface of the polymeric base; and
(c) curing the polymeric base until the polymeric materials have substantially completed polymerization thereby securing the particles into the polymeric base.

61. The method of making a polymeric material with a biocompatible particulate surface of claim 60 wherein the particles are particles including one or more biocompatible purified proteins interacting with one or more biocompatible solvents.

62. The method of making a polymeric material with a biocompatible particulate surface of claim 60 wherein the particles have a size of approximately 1 μm to 1000 μm.

63. The method of making a polymeric material with a biocompatible particulate surface of claim 61 wherein the biocompatible proteins are selected from the group consisting of elastin, collagen, albumin, keratin, laminin, fibronectin, silk, silk fibroin, actin, myosin, fibrinogen, thrombin, aprotinin, antithrombin III, elastinlike blocks, silklike blocks, collagenlike blocks, lamininlike blocks, fibronectinlike blocks and silklike, elastinlike blocks, collagen-heparin, collagen-elastin-heparin and collagen-chondroiten.

64. The method of making a polymeric material with a biocompatible particulate surface of claim 61 wherein the biocompatible solvent is selected from the group consisting of water, dimethyl sulfoxide (DMSO), biocompatible alcohols, biocompatible acids, oils and biocompatible glycols.

65. The method of making a polymeric material with a biocompatible particulate surface of claim 61 wherein the biocompatible particles further include one or more pharmacologically active agents selected from the group consisting of analgesics, anesthetics, antipsychotic agents, angiogenic growth factors, bone mending biochemicals, steroids, antisteroids, corticosteroids, antiglacoma agents, antialcohol agents, anti-coagulants agents, genetic material, antithrombolytic agents, anticancer agents, anti-Parkinson agents, antiepileptic agents, permeation enhancers, anti-inflammatory agents, anticonception agents, enzymes agents, cells, growth factors, antiviral agents, antibacterial agents, antifungal agents, hypoglycemic agents, antihistamine agents, chemoattractants, neutraceuticals, antiobesity, smoking cessation agents, obstetric agents and antiasmatic agents.

66. The method of making a polymeric material with a biocompatible particulate surface of claim 61 further comprising one or more biocompatible additives selected from the group consisting of epoxies, polyesters, acrylics, nylons, silicones, polyanhydride, polyurethane, polycarbonate, poly(tetrafluoroethylene), polycaprolactone, polyethylene oxide, polyethylene glycol, poly(vinyl chloride), polylactic acid, polyglycolic acid, polypropylene oxide, poly(akylene)glycol, polyoxyethylene, sebacic acid, polyvinyl alcohol, 2-hydroxyethyl methacrylate, polymethyl methacrylate, 1,3-bis(carboxyphenoxy)propane, lipids, phosphatidylcholine, triglycerides, humectants, polyhydroxybutyrate, polyhydroxyvalerate, poly(ethylene oxide), poly ortho esters, poly (amino acids), polycyanoacrylates, polyphophazenes, polysulfone, polyamine, poly (amido amines), fibrin, graphite, flexible fluoropolymer, isobutyl-based, isopropyl styrene, vinyl pyrrolidone, cellulose acetate dibutyrate, silicone rubber, and copolymers or combinations of these.
